(19) United States
(12) Patent Application Publication
(10) Pub. No.: US 2013/0259899 A1
(43) Pub. Date: Oct. 3, 2013

(54) DETECTION OF FUSOBACTERIUM IN A GASTROINTESTINAL SAMPLE TO DIAGNOSE GASTROINTESTINAL CANCER

(71) Applicants: Emma Allen-Vercoe, Guelph (CA); Robert Holt, North Vancouver (CA); Richard Moore, Vancouver (CA); Rene Warren, Vancouver (CA)

(72) Inventors: Emma Allen-Vercoe, Guelph (CA); Robert Holt, North Vancouver (CA); Richard Moore, Vancouver (CA); Rene Warren, Vancouver (CA)

(73) Assignees: ALLEN-VERCOE, EMMA MOLECULAR AND CELLULAR BIOLOGY UNIVERSITY OF GUELPH, Guelph, ON (CA); BRITISH COLOMBIA CANCER AGENCY BRANCH, Vancouver, BC (CA)

(21) Appl. No.: 13/877,421

(22) PCT Filed: Oct. 4, 2011
(86) PCT No.: PCT/CA11/01108

§ 371 Date: Jun. 12, 2013

Related U.S. Application Data
(60) Provisional application No. 61/389,404, filed on Oct. 4, 2010.

(51) Int. Cl. C12Q 1/68

(52) U.S. Cl. CPC C12Q 1/6886
USPC 424/234.1; 435/6.11; 435/32; 435/7.1; 435/7.9

(57) ABSTRACT

Fusobacterium is a genus of gram-negative, filamentous, anaerobic bacteria found as normal flora in the mouth and large bowel, and often in necrotic tissue. A comparison of microbial ribonucleic acids (RNA) between colorectal carcinoma (CRC) tissue and adjacent normal control tissue found the over-representation of F. nucleatum in CRC tissue. RNA abundance was measured by polymerase chain reaction (PCR)-amplifying RNA from the tissue, constructing libraries, sequencing the RNA in the libraries, pairing sequences from CRC and normal tissue, and quantifying RNA abundance. Detection of Fusobacterium in a gastrointestinal sample is indicative of gastrointestinal cancer.

### FIELD OF INVENTION

The present invention provides, in part, methods for diagnosis or treatment of gastrointestinal cancers.

### BACKGROUND OF THE INVENTION

Cancers of the gastrointestinal tract represent a significant percentage of all cancer related deaths, and include gastric cancer, colorectal and esophageal cancers. Colorectal carcinoma (CRC) is the second leading cause of cancer deaths, responsible for approximately 655,000 deaths per year worldwide (World Health Organization fact sheet #297, February 2009). CRC is also one of the first and best genetically characterized cancers in which specific somatic mutations on oncogenes and tumour suppressor genes associated with progression from adenomatous lesions (polyps) to invasive carcinoma have been identified (Vogelstein et al. 1988). Inflammation has been recognized as a risk factor for CRC (McLean et al. 2011, Wu et al. 2009).

Infectious agents have been implicated in the development of some cancers (Herrera L A et al. 2005). Among these, Human Papilloma Virus (cervical cancer), Hepatitis B and C virus (liver cancer), and Helicobacter pylori (gastric cancer) alone are responsible for an estimated 15% of the global cancer burden, based on strength of the association and prevalence of infection (Parkin et al. 2006).

Fusobacterium nucleatum is an invasive (Han et al. 2000, Swidsinski et al. 2011), adherent (Weiss et al. 2000) and pro-inflammatory (Peyret-Lacombe et al. 2009, Krisanaprakornkit et al. 2000) anaerobic bacterium. It is common in dental plaque (Bolstad et al. 1996, Ximenez-Fyvie et al. 2000) and there is a well established association between F. nucleatum and periodontitis (Signal et al. 2011). Anecdotally, F. nucleatum has been implicated in cerebral abscesses (Kai et al. 2008) and pericarditis (Han et al. 2003) and it is one of the Fusobacterium species implicated in Lemierre's syndrome, a rare form of thrombophlebitis (Weeks et al. 2010). Various Fusobacteria, including F. nucleatum, have been implicated in acute appendicitis, where they have been found by immunohistochemistry (IHC) as epithelial and submucosal infiltrates that correlate positively with severity of disease (Swidsinski et al. 2011). When isolated from human intestinal biopsy material, F. nucleatum has been found to be more readily culturable from patients with gastrointestinal (GI) disease than healthy controls, and the strains grown from inflamed biopsy tissue appeared to exhibit a more invasive phenotype (Strauss et al. 2008, Strauss et al. 2011).

### SUMMARY OF THE INVENTION

The present invention provides, in part, methods for diagnosis or treatment of gastrointestinal cancers.

In one aspect, the invention provides a method for prognosing or diagnosing a gastrointestinal cancer in a subject, by providing a sample from the subject; and detecting a Fusobacterium sp. in the sample, where a positive detection of the Fusobacterium sp. indicates a prognosis or diagnosis of gastrointestinal cancer.

In some embodiments, the detection may include contacting the sample with an antibody that specifically binds a Fusobacterium sp. antigen or a nucleotide sequence that hybridizes to a Fusobacterium sp. nucleotide sequence, where the specific binding of the antibody to the Fusobacterium sp. antigen or the hybridization of the nucleotide sequence to the Fusobacterium sp. nucleotide sequence indicates a prognosis or diagnosis of gastrointestinal cancer.

The Fusobacterium sp. antigen or nucleotide sequence may be selected from the group consisting of one or more of the polypeptides descried herein.

The gastrointestinal cancer may be a colorectal carcinoma.

The subject may have or may be suspected of having chronic inflammatory bowel disease. The subject may be a human.

The sample may be a colon sample, a rectal sample, a stool sample, an adenomatous lesion or polyp, or may be derived from an abscess.

The Fusobacterium sp. may be a F. nucleatum.

In alternative aspects, the invention provides a method of screening for a compound for treating a gastrointestinal cancer, by providing a test compound; and determining whether the test compound inhibits the growth or activity of a Fusobacterium sp., where a compound that inhibits the growth or activity of the Fusobacterium sp. is a candidate compound for treating a gastrointestinal cancer.

In alternative aspects, the invention provides a method of treating a gastrointestinal cancer, by administering a compound or composition that induces an immunological response against a Fusobacterium sp. to a subject diagnosed with or suspected of having a gastrointestinal cancer.

This summary of the invention does not necessarily describe all features of the invention. Further aspects of the invention will become apparent from consideration of the ensuing description. A person skilled in the art will realise that other embodiments of the invention are possible and that the details of the invention can be modified in a number of respects, all without departing from the inventive concept. Thus, the following drawings, descriptions and examples are to be regarded as illustrative in nature and not restrictive.

### BRIEF DESCRIPTION OF THE DRAWINGS

These and other features of the invention will become more apparent from the following description in which reference is made to the appended drawings wherein:

FIG. 1 is a schematic diagram showing a strategy for detection of candidate infectious agents;

FIG. 2 shows the detection of HVP DNA sequences in normal and tumour samples;

FIG. 3A shows the detection of Fusobacterium DNA sequences;

FIG. 3B shows the number of sequencing read pairs that match known microbial genomes for the 25 most abundant genomes.

FIG. 4A shows the relative abundance of Fusobacterium in tumour versus normal colorectal carcinoma biopsies. Relative amounts of Fusobacterium DNA were determined between tumour and matched normal biopsies in 99 subjects, using quantitative real-time PCR (qPCR). The cycle threshold (Ct) values for the normal samples had a Ct range of 25.5 to 40 and the Ct range for the tumour samples was between 21.4 to 40. Data shown are mean values from two independent experiments. Fusobacterium load, as determined by qPCR, was found to be significantly higher in the tumour samples versus the matched control samples (two-tailed ratio t-test (p=2.52×10-6)).

FIG. 4B shows the detection of Fusobacterium DNA sequences by qPCR in cohort of 90 patients using matched normal and tumor samples.

FIGS. 5A-B show the frequency of metastasis increases with higher Fusobacterium abundance in tumour biopsies. Patients with 5× or greater Fusobacterium in their tumour biopsies versus matched normal tissue were compared to those patients with less than 5× relative amounts of Fusobacterium. A significantly higher number of patients from the high Fusobacterium group (A) had more tumour spreading in their lymph nodes as measured by their surgical TNM scores than the low Fusobacterium group (B) (one-tailed Fisher's exact test p-value=0.0035).

### DETAILED DESCRIPTION

The present invention provides, in part, methods for diagnosis or treatment of gastrointestinal cancers by detection of Fusobacterium. We disclose herein that Fusobacterium nucleatum, a known pathogen associated previously with periodontal disease, is associated with gastrointestinal cancer. We also demonstrate that a Fusobacterium isolated from CRC tumour samples is invasive.

More specifically, we used a metagenomics approach (Weber et al. 2002, Moore et al. 2011) to identify microbial sequence signatures in diseases that have a possible or suspected infectious etiology. There are variations on the method, but the basic approach involves shotgun sequencing bulk DNA or RNA isolated from disease tissue, computational subtraction of all sequence reads recognized as human, and comparison of the residual reads to databases of known microbial sequences in order to identify microbial species present in the initial specimen. The method is complementary to traditional culture and histolology based protocols and new, massively parallel sequencing technologies impart high sensitivity. Such methods are described in part in, for example, Flicek et al. 2011, Li and Durbin 2010, Moore et al. 2011, etc.

More specifically, we screened colorectal carcinoma and matched normal tissue specimens using RNA-Seq, followed by host sequence subtractions, and found marked over-representation of F. nucleatum sequences in tumours relative to control specimens. We obtained a Fusobacterium isolate from a frozen tumour specimen and this showed highest sequence similarity to a gut mucosa isolate and was confirmed to be invasive. We verified overabundance of Fusobacterium sequences in tumour versus matched normal control tissue by quantitative PCR analysis from a total of 99 subjects (p=2.5E-6) and observed a positive association with lymph node metastasis.

This over-representation of F. nucleatum in colorectal tumour specimens was largely unexpected, given that it is generally regarded as an oral pathogen, and is not an abundant constituent of the normal gut microbiota (Qin, et al. 2010).

By a “cancer” or “neoplasm” is meant any unwanted growth of cells serving no physiological function. In general, a cell of a neoplasm has been released from its normal cell division control, i.e., a cell whose growth is not regulated by the ordinary biochemical and physical influences in the cellular environment. In most cases, a neoplastic cell proliferates to form a clone of cells which are either benign or malignant. Examples of cancers or neoplasms include, without limitation, transformed and immortalized cells, tumours, and carcinomas such as breast cell carcinomas and prostate carcinomas. The term cancer includes cell growths that are technically benign but which carry the risk of becoming malignant i.e. a “malignancy.” By “malignancy” is meant an abnormal growth of any cell type or tissue. The term malignancy includes cell growths that are technically benign but which carry the risk of becoming malignant. This term also includes any cancer, carcinoma, neoplasm, neoplasia, or tumor. Identification and classification of types and stages of cancers may be performed by using for example information provided by the Surveillance, Epidemiology, and End Results (SEER) Program of the National Cancer Institute.

By “gastrointestinal” or “GI” cancer or carcinoma is meant a malignancy or neoplasm of the gastrointestinal tract. GI cancers can include cancers of the upper GI tract such as, esophagus (e.g., squamous cell carcinoma, adenocarcinoma), or stomach (e.g., gastric carcinoma, signet ring cell carcinoma, gastric lymphoma) or of the lower GI tract such as, small intestine (e.g., duodenal cancer/adenocarcinoma), colon/rectum (e.g., colorectal polyps/Peutz-Jeghers syndrome, juvenile polyposis syndrome, familial adenomatous polyposis/Gardner's syndrome, Cronkhite-Canada syndrome, familial adenomatous polyposis, hereditary nonpolyposis colorectal cancer, etc.), anus (e.g., squamous cell carcinoma).

In some embodiments, the methods and compounds described or referenced herein may pertain to a condition or a cancer that is “related” to a GI cancer. Such cancers can include, for example, liver cancer or pancreatic cancer, or a cancer of a tissue or organ to which a colorectal tumour or cell has spread by metastasis. In alternative embodiments, conditions such as abscesses in other tissues, such as liver, are included.

By “Fusobacterium” is meant a genus of gram-negative, anaerobic, rod-shaped bacteria found as normal flora in the mouth and large bowel and often in necrotic tissue (Miller-Keane Encyclopedia and Dictionary of Medicine, Nursing, and Allied Health, Seventh Edition. © 2003 by Saunders, an imprint of Elsevier, Inc.). Some Fusobacterium species are pathogenic to humans (Mosby's Medical Dictionary, 8th edition. © 2009, Elsevier). Fusobacterium species include F. gonidiaformans and F. mortiferum (occurring in respiratory, urogenital, and gastrointestinal infections); F. necrophorum (occurring in disseminated infections involving necrotic lesions, abscesses, and bacteremia), F. naviforme, F. russii, and F. varium (occurring in abscesses and other infections), F. fusiforme (found in cavities of humans and other animals, and sometimes associated with Vincent's angina), F. polymorphum, F. equinum, F. nodosus, F. nucleatum, etc. (Miller-Keane Encyclopedia and Dictionary of Medicine, Nursing, and Allied Health, Seventh Edition. © 2003 by Saunders, an imprint of Elsevier, Inc.; Mosby's Medical Dictionary, 8th edition. © 2009, Elsevier). In some embodiments, a Fusobacterium species includes a Fusobacterium sp. strain 3_{—}1_{—}36A2, Fusobacterium sp. strain 3_{—}1_{—}27, Fusobacterium sp. strain 7_{—}1, Fusobacterium sp. strain 4_{—}1_{—}13, Fusobacterium sp. strain D11, Fusobacterium sp. strain 3_{—}1_{—}33, F. gonidiaformans ATCC 25563, Fusobacterium sp. strain 1_{—}1_{—}41FAA, etc.

By “Fusobacterium nucleatum” or “F. nucleatum” is meant an invasive, adherent and pro-inflammatory anaerobic bacterium. In some embodiments, a F. nucleatum includes a F. nucleatum subsp. nucleatum ATCC 25586, F. nucleatum subsp. polymorphum ATCC 10953, Fusobacterium sp. strain 3_{—}1_{—}36A2, F. nucleatum CC53, Fusobacterium sp. strain 3_{—}1_{—}27, F. nucleatum subsp. vincentii ATCC 49256, Fusobacterium sp. strain 7_{—}1, Fusobacterium sp. strain 4_{—}1_{—}13, Fusobacterium sp. strain D11, F. nucleatum subsp. nucleatum ATCC 23726, Fusobacterium sp. strain 3_{—}1_{—}33, Fusobacterium sp. strain 1_{—}1_{—}41FAA, etc.

In some embodiments, the F. nucleatum subsp. nucleatum ATCC 25586 has a nucleic acid sequence substantially identical to one or more of the sequences referenced in GenBank Accession No. AE009951 or to NC_{—}003454.1 or a fragment or variant thereof. In some embodiments, the F. nucleatum subsp. polymorphum ATCC 10953 has a nucleic acid sequence substantially identical to one or more of the sequences referenced in GenBank Accession No. NZ_CM000440, or a fragment or variant thereof. In some embodiments, the Fusobacterium sp. strain 3_{—}1_{—}36A2 has a nucleic acid sequence substantially identical to one or more of the sequences referenced in GenBank Accession Nos. ACPU01000001 to ACPU01000051, or GG698790-GG698801, or a fragment thereof. In some embodiments, a Fusobacterium sequence according to the invention has a nucleic acid sequence substantially identical to one or more of the sequences listed in Table 4, or encodes a polypeptide as described in Table 4, or other sequences described or referenced herein, or fragments or variants thereof.

The terms “nucleic acid” or “nucleic acid molecule” encompass both RNA (plus and minus strands) and DNA, including cDNA, genomic DNA (gDNA), and synthetic (e.g., chemically synthesized) DNA. The nucleic acid may be double-stranded or single-stranded. Where single-stranded, the nucleic acid may be the sense strand or the antisense strand. A nucleic acid molecule may be any chain of two or more covalently bonded nucleotides, including naturally occurring or non-naturally occurring nucleotides, or nucleotide analogs or derivatives. By “RNA” is meant a sequence of two or more covalently bonded, naturally occurring or modified ribonucleotides. One example of a modified RNA included within this term is phosphorothioate RNA. By “DNA” is meant a sequence of two or more covalently bonded, naturally occurring or modified deoxyribonucleotides. By “cDNA” is meant complementary or copy DNA produced from an RNA template by the action of RNA-dependent DNA polymerase (reverse transcriptase). Thus a “cDNA clone” means a duplex DNA sequence complementary to an RNA molecule of interest, carried in a cloning vector. By “complementary” is meant that two nucleic acids, e.g., DNA or RNA, contain a sufficient number of nucleotides which are capable of forming Watson-Crick base pairs to produce a region of double-strandedness between the two nucleic acids. Thus, adenine in one strand of DNA or RNA pairs with thymine in an opposing complementary DNA strand or with uracil in an opposing complementary RNA strand. It will be understood that each nucleotide in a nucleic acid molecule need not form a matched Watson-Crick base pair with a nucleotide in an opposing complementary strand to form a duplex. A nucleic acid molecule is “complementary” to another nucleic acid molecule if it hybridizes, under conditions of high stringency, with the second nucleic acid molecule.

A “substantially identical” sequence is an amino acid or nucleotide sequence that differs from a reference sequence only by one or more conservative substitutions, as discussed herein, or by one or more non-conservative substitutions, deletions, or insertions located at positions of the sequence that do not destroy the biological function of the amino acid or nucleic acid molecule. Such a sequence can be any value from 50% to 99%, or more generally at least 50% 55% or 60%, or at least 65%, 75%, 80%, 85%, 90%, or 95%, or as much as 96%, 97%, 98%, or 99% identical when optimally aligned at the amino acid or nucleotide level to the sequence used for comparison using, for example, the Align Program (Myers and Miller, CABIOS, 1989, 4:11-17) or FASTA. For polypeptides, the length of comparison sequences may be at least 2, 5, 10, or 15 amino acids, or at least 20, 25, or 30 amino acids. In alternate embodiments, the length of comparison sequences may be at least 35, 40, or 50 amino acids, or over 60, 80, or 100 amino acids. For nucleic acid molecules, the length of comparison sequences may be at least 5, 10, 15, 20, or 25 nucleotides, or at least 30, 40, or 50 nucleotides. In alternate embodiments, the length of comparison sequences may be at least 60, 70, 80, or 90 nucleotides, or over 100, 200, or 500 nucleotides. Sequence identity can be readily measured using publicly available sequence analysis software (e.g., Sequence Analysis Software Package of the Genetics Computer Group, University of Wisconsin Biotechnology Center, 1710 University Avenue, Madison, Wis. 53705, or BLAST software available from the National Library of Medicine, or as described herein). Examples of useful software include the programs Pile-up and PrettyBox. Such software matches similar sequences by assigning degrees of homology to various substitutions, deletions, substitutions, and other modifications.

Alternatively, or additionally, two nucleic acid sequences may be “substantially identical” if they hybridize under high stringency conditions. In some embodiments, high stringency conditions are, for example, conditions that allow hybridization comparable with the hybridization that occurs using a DNA probe of at least 500 nucleotides in length, in a buffer containing 0.5 M NaHPO₄, pH 7.2, 7% SDS, 1 mM EDTA, and 1% BSA (fraction V), at a temperature of 65° C., or a buffer containing 48% formamide, 4.8×SSC, 0.2 M Tris-Cl, pH 7.6, 1×Denhardt's solution, 10% dextran sulfate, and 0.1% SDS, at a temperature of 42° C. (These are typical conditions for high stringency northern or Southern hybridizations.) Hybridizations may be carried out over a period of about 20 to 30 minutes, or about 2 to 6 hours, or about 10 to 15 hours, or over 24 hours or more. High stringency hybridization is also relied upon for the success of numerous techniques routinely performed by molecular biologists, such as high stringency PCR, DNA sequencing, single strand conformational polymorphism analysis, and in situ hybridization. In contrast to northern and Southern hybridizations, these techniques are usually performed with relatively short probes (e.g., usually about 16 nucleotides or longer for PCR or sequencing and about 40 nucleotides or longer for in situ hybridization). The high stringency conditions used in these techniques are well known to those skilled in the art of molecular biology, and examples of them can be found, for example, in Ausubel et al., Current Protocols in Molecular Biology, John Wiley & Sons, New York, N.Y., 1998, which is hereby incorporated by reference. A nucleic acid sequence may be detectably labelled.

Substantially identical sequences may for example be sequences that are substantially identical to the Fusobacterium species sequences described or referenced herein, or fragments or variants thereof.

An antibody “specifically binds” an antigen when it recognises and binds the antigen, for example, an antigen from a Fusobacterium, such as a F. nucleatum, but does not substantially recognise and bind other molecules in a sample, for example, an antigen from a different species. Such an antibody has, for example, an affinity for the antigen which is at least 10, 100, 1000 or 10000 times greater than the affinity of the antibody for another reference molecule in a sample. An antibody may be detectably labelled.

By “detectably labelled” is meant any means for marking and identifying the presence of a molecule, e.g., an oligonucleotide probe or primer, a gene or fragment thereof, or a cDNA molecule. Methods for detectably-labelling a molecule are well known in the art and include, without limitation, radioactive labelling (e.g., with an isotope such as ³²P or ³⁵S) and nonradioactive labelling such as, enzymatic labelling (for example, using horseradish peroxidase or alkaline phosphatase), chemiluminescent labeling, fluorescent labeling (for example, using fluorescein), bioluminescent labeling, or antibody detection of a ligand attached to the probe. Also included in this definition is a molecule that is detectably labeled by an indirect means, for example, a molecule that is bound with a first moiety (such as biotin) that is, in turn, bound to a second moiety that may be observed or assayed (such as fluorescein-labeled streptavidin). Labels also include digoxigenin, luciferases, and aequorin.

A “sample” can be any organ, tissue, cell, or cell extract isolated from a subject, such as a sample isolated from a mammal having a gastrointestinal cancer or suspected of having a gastrointestinal cancer. For example, a sample can include, without limitation, cells or tissue (e.g., from a biopsy or autopsy) from any part of the gastrointestinal tract (including without limitation, colon, stomach, stool, anus, rectum, duodenum), a gastrointestinal cell lysate, cell culture or culture medium, or any other specimen, or any extract thereof, obtained from a patient (human or animal), test subject, or experimental animal. A sample may also include, without limitation, products produced in cell culture by normal or transformed cells (e.g., via recombinant DNA or monoclonal antibody technology). A sample may also include, without limitation, any organ, tissue, cell, or cell extract isolated from a non-mammalian subject, such as an insect or a worm. A “sample” may also be a cell or cell line created under experimental conditions, that is not directly isolated from a subject. A sample can also be cell-free, artificially derived or synthesised. A sample may be from a gastrointestinal cell or tissue known to be cancerous, suspected of being cancerous, or believed not be cancerous (e.g., normal or control). In some embodiments, an oral sample is specifically excluded.

A “subject” may be a human, non-human primate, rat, mouse, cow, horse, pig, sheep, goat, dog, cat, etc. The subject may be a clinical patient, a clinical trial volunteer, an experimental animal, etc. The subject may be suspected of having or at risk for having a GI cancer or related condition or cancer, be diagnosed with a GI cancer or related condition or cancer, or be a control subject that is confirmed to not have a GI cancer or related condition or cancer. Diagnostic methods for GI cancer or related condition or cancer and the clinical delineation of such diagnoses are known to those of ordinary skill in the art.

The association of an invasive Fusobacterium with a GI cancer, such as CRC, permits the use of this association for screening methods. Such screens may be performed using assays as described herein or known in the art.

In alternative aspects, a GI cancer or related condition or cancer may be treated by administering an effective amount of a compound (e.g., an antibiotic) or a composition (e.g., a vaccine) effective against a Fusobacterium, such as a F. nucleatum. In some embodiments, a vaccine may include a Fusobacterium or antigen thereof (e.g., a polypeptide encoded by one or more of the Fusobacterium sequences described or referenced herein, or known in the art, or a whole bacterium, such as a killed Fusobacterium bacterin). In the case of vaccine formulations, an immunogenically effective amount of a compound of the invention can be provided, alone or in combination with other compounds, with an immunological adjuvant, for example, Freund's incomplete adjuvant, dimethyldioctadecylammonium hydroxide, or aluminum hydroxide. The compound may also be linked with a carrier molecule, such as bovine serum albumin or keyhole limpet hemocyanin to enhance immunogenicity.

In alternative embodiments, such compounds, compositions or vaccines be combined with more traditional and existing therapies for GI cancer or related condition or cancer.

An “effective amount” includes a therapeutically effective amount or a prophylactically effective amount. A “therapeutically effective amount” refers to an amount effective, at dosages and for periods of time necessary, to achieve the desired therapeutic result, such as treatment of a GI cancer or related condition or cancer. A therapeutically effective amount of a compound may vary according to factors such as the disease state, age, sex, and weight of the individual, and the ability of the compound to elicit a desired response in the individual. Dosage regimens may be adjusted to provide the optimum therapeutic response. A therapeutically effective amount is also one in which any toxic or detrimental effects of the compound are outweighed by the therapeutically beneficial effects. A “prophylactically effective amount” refers to an amount effective, at dosages and for periods of time necessary, to achieve the desired prophylactic result, such prophlaxis of a GI cancer or related condition or cancer. Typically, a prophylactic dose is used in subjects prior to or at an earlier stage of disease, so that a prophylactically effective amount may be less than a therapeutically effective amount.

### Example 1

### Fusobacterium Nucleatum Infection is Prevalent in Human Colorectal Carcinoma

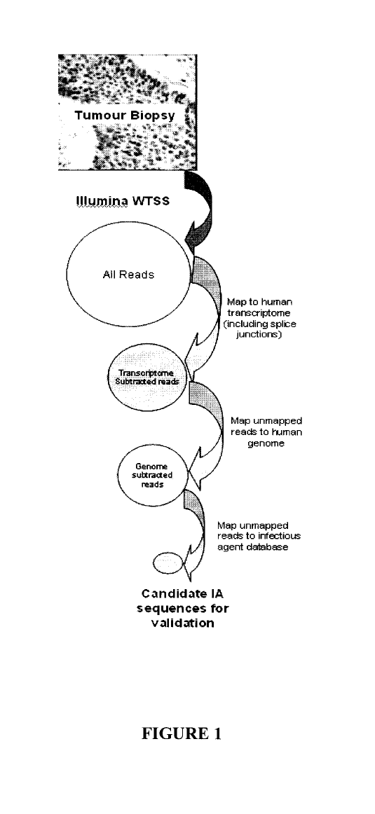

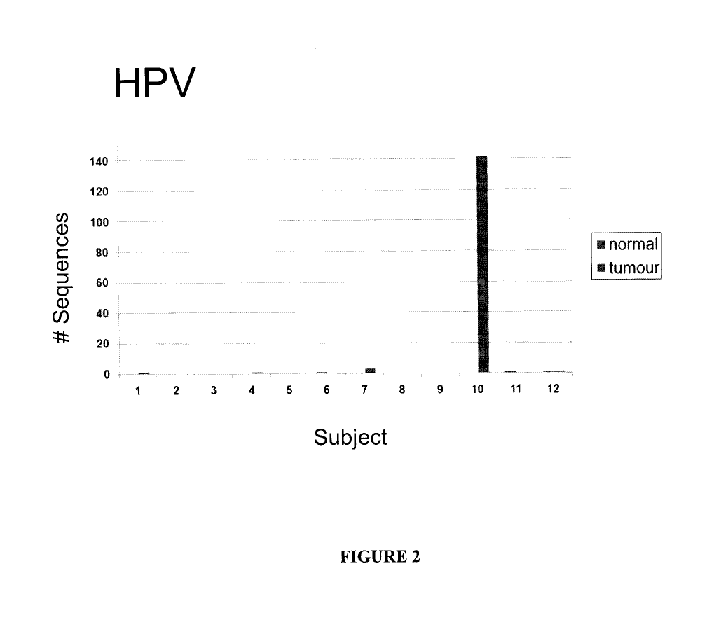

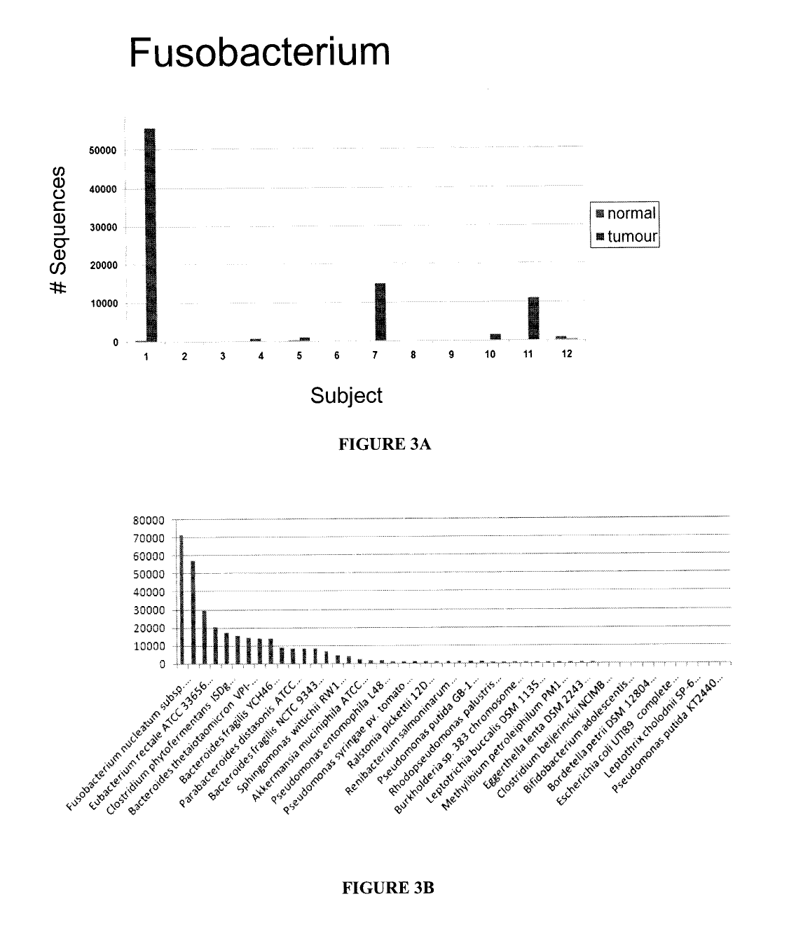

Deep transcriptome sequencing of tumour/normal specimens from 12 subjects was performed using Illumina platform at the Genome Sciences Center at the BC Cancer Agency, as outlined in FIG. 1. Validation studies were performed using qPCR to test matched and normal pairs of specimens from colorectal carcinoma subjects. HPV virus was detected in one of the twelve patients (FIG. 2). Fusobacterium was detected in four of the twelve patient samples (FIG. 3A).

More specifically, total RNA was isolated from frozen sections of eleven matched pairs of CRC and adjacent normal tissue specimens. RNA was purified by host ribosomal sequence depletion, rather than poly-A selection, in order to retain non-polyadenylated sequences of potential microbial origin. In our screen, we analyzed RNA rather than DNA in order to detect active, transcribing microorganisms and to allow for the detection of RNA viruses that may be present.

For all cases, fresh CRC samples were obtained with informed consent by the BC Cancer Agency Tumour Tissue Repository (BCCA-TTR), which operates as a dedicated biobank with approval from the University of British Columbia-British Columbia Cancer Agency Research Ethics Board (BCCA REB). The BCCA-TTR platform are governed by Standard Operating Procedures (SOPs) that meet or exceed the recommendations of international best practice guidelines for repositories (NCl Office of Biorepositories and Biospecimen Research. NCl Best Practices for Biospecimen Resources. 2007). Specimens are handled with very close attention to maintaining integrity and isolation. Overall average collection time (time from removal from surgical field to cryopreservation in liquid nitrogen) for all colorectal cases in the BCCA-TTR is 31 min. For this study, biospecimens were held briefly at −20° C. during frozen sectioning, using 100% ethanol to clean the blade between all samples. Clinical-pathological and outcomes data was obtained from the BC Cancer Agency clinical chart including tumour features reported according to the American College of Pathologists criteria and ‘Protocol for examination of specimens from patients with primary carcinoma of the colon and rectum’. This included histological features indicative of inflammatory and immune response (lymphoid and myeloid cell infiltrates) which were assessed as none, mild-moderate, or marked using semi-quantitative scoring as well as the percent area of tumour involved by necrosis, by a pathologist in a representative tumour cross section.

Illumina RNASeq libraries were constructed, barcoded, and pooled, and 2 lanes of paired end sequencing data were obtained using the Illumina GAIIx platform. Reads were filtered for base quality and low complexity, then aligned pairwise to human rRNA and cDNA and genome (hg18) reference sequences using Burrows-Wheeler Aligner (BWA). Aligned reads were removed from the data set, leaving 34.9M pairs (Table 1).

TABLE 1
Host sequence subtraction RNA-seq data from eleven colorectal carcinoma
and matched normal specimens.

| | Control (mean +/− SD) | Tumour (mean +/− SD) |
|---|---|---|
| Raw read pairs | 2,222,539 +/− 355,530 | 2,175,063 +/− 439,279 |
| Filtered read pairs¹ | 349,354 +/− 212,209 | 339,935 +/− 182,207 |
| Read pairs matching | 17,154 +/− 22,837 | 15,681 +/− 29,568 |
| bacterial or viral | | |
| genomes² | | |
| Distinct bacterial or | 546 | 544 |
| viral genome | | |
| matches | | |

¹Read pairs remaining after removal of low quality reads and reads matching human RNA, transcriptome or genome reference sequences.
²Unambiguous alignments where the best match for each mate pair is to the same accession.

These residual read pairs were then used to search a custom database containing accessions for all Refseq bacterial and viral genomes, using Novoalign (http://novocraft.com), which is a slower but more permissive aligner than BWA. Our analysis was alignment based, because the abundance of candidate organisms can be inferred more directly from alignments than from de novo assemblies. For accuracy, we tallied only unambiguous alignments where the best match to both the forward and reverse mate pair was to the same genome accession.

More specifically, eleven colorectal tumour samples and eleven matched normal samples were processed, using an RNeasy Plus mini kit (Qiagen) to purify total RNA or an AllPrep DNA/RNA mini kit (Qiagen) to purify both DNA and RNA. RNA quality and concentration was assessed using Agilent Bioanalyzer 2000 RNA Nanochips. Ribosomal RNAs were depleted from 1 mg of total RNA using the manufacturer's protocol for the RiboMinus Eukaryote Kit for RNA-Seq (Invitrogen). Depletion was assessed using Agilent Bioanalyzer 2000 RNA Nanochips. All samples were found to have <10% residual ribosomal RNA contamination and were processed as described previously (Shah et al. 2009, Morin et al. 2010) for the construction of Illumina libraries, with the following modifications: Each paired-end library was PCR amplified for 15 cycles using the standard Illumina PE1 PCR primer plus one of 12 modified PE2 primers, each including a unique six base insertion as an index sequence. Libraries prepared using indexed primers were then combined in pools of 11 each (one tumour pool, one control pool) gel purified, and then sequenced on the Illumina GAIIx platform. One lane of 75 bp paired end sequence was obtained for each of the two pools.

Paired-end sequence reads from indexed tumour and adjacent normal sample libraries were processed. Briefly, corresponding human RNA-Seq libraries were aligned with BWA (version 0.5.4 [sample-o 1000, default options] sequentially against human rRNA, cDNA and genome reference sequences. Pairs aligning logically, or containing reads having either an average base quality below phred 20 (Ewing et al. 1998) and/or more than 20 consecutive homopolymeric bases were subtracted from the original data. Read pairs that remained unaligned to any of the human sequence databases were used to interrogate a custom-built sequence collection of well-characterized bacterial and viral genes and genomes using novoalign (version 2.05.20 [-o SAM-r A-R 0, default options]). Alignments were run on a single 3 GHz 8 CPU Intel® Xeon® 64-bit 61 GB RAM computer running CentOS release 5.4. Multiplexed reads from the tumour and normal libraries were deconvoluted according to sequence tags (i.e., barcodes) and the number of read pairs that mapped unambiguously to a single location were tallied for each indexed sample and normalized against the sample read count. Ultimately, read pair count was reported for each GenBank accession in our microbial genome database, sorted in decreasing order by the sum of unambiguous pairs and PERL scripts were developed to mine these data. Read counts were graphically visualized first by clustering common accession reads using UPGMA (Sokal and Michener, 1958) and then displayed as a heat map (log 10 scale) using the Mayday package (http://www-ps.informatik.uni-tuebingen.de/mayday/wp/).

These alignments identified a total of 670 distinct genome accessions, representing 415 species. These were predominantly (97%) bacterial, although several herpes virus sequences were detectable at low levels, and one tumour showed overabundance (142 raw read pairs) of human papillomavirus type 107 (GenBank accession EF422221.1). A wide distribution of bacterial species abundance was apparent, with 30 species representing 95% of the sequence data, with twenty five of the most abundant genomes shown in FIG. 3B. Of the 670 distinct genome accessions hit, 63% were found in both tumour and normal specimens. Alignments specific to only tumour or only control specimens were due to rare sequences and, therefore, the representation in one group or the other may simply reflect sampling bias. Markedly disproportionate alignments between tumour and control were to the genome of F. nucleatum subsp. nucleatum (ATCC 25586), a Gram-negative anaerobe. F. nucleatum was the organism with the highest number of hits overall (21% of all alignments) and nine of the eleven subjects showed at least two-fold higher read counts in tumour relative to corresponding control tissue.

The abundance of normalized bacterial read pairs ranged from zero to a maximum of 66,896. Differential abundance ranged from 0.1 to 256-fold, with a mean over-abundance of 79-fold. The majority of the hits were to highly abundant F. nucleatum ribosomal transcripts but other non-ribosomal F. nucleatum gene products were also detected. More specifically, the distribution of hits from colorectal carcinoma RNA-Seq data to the annotated F. nucleatum subsp. nucleatum ATCC 25586 genome showed 87% of the hits to be to LSU Ribosomal RNA 731537-734463, 9% of the hits to SSU Ribosomal RNA, 1% to LSU Ribosomal RNA 1073886-1076812, and the remaining 3% to other hits with more than 10 pairs (e.g., hypothetical protein FN0264, hypothetical protein FN1792, Asn tRNA, SSU Ribosomal RNA elongation factor Tu, pyruvate-flavodoxin oxidoreductase, protein translation elongation factor G (EF-G), acyl carrier protein, hypothetical protein FN1314, SSU ribosomal protein S6P, CIpB protein, SSU ribosomal protein S10P, putative cytoplasmic protein, preprotein translocase subunit SecY, 50S ribosomal protein L31P, 50S ribosomal protein L32P, 50S ribosomal protein L33P, protein translation initiation factor 1, DNA-directed RNA polymerase subunit alpha, 50S ribosomal protein L3P, 50S ribosomal protein L2, 50S ribosomal protein L1, flavodoxin FldA, hypothetical protein FN1309, 30S ribosomal protein S2, 30S ribosomal protein S4, major outer membrane protein, alkyl hydroperoxide reductase C22 protein, 50S ribosomal protein L10P, Glu tRNA, 50S ribosomal protein L28P, HD superfamily hydrolase, hypothetical protein FN1118, 5S ribosomal RNA, 50S ribosomal protein L21P, 50S ribosomal protein L13, thioredoxin, 50S ribosomal protein L11P, 50S ribosomal protein L19, 60 kDa chaperonin GROEL, transcription antitermination protein nusG, 50S ribosomal protein L18P, 50S ribosomal protein L217P, Leu tRNA, 50S ribosomal protein L4, 50S ribosomal protein L14P, carbon starvation protein A, SSU ribosomal protein S9P, DNA-directed RNA polymerase beta chain, 30S ribosomal protein S12, SSU ribosomal protein S3P). The total number of read pair hits was 80,118.

### Example 2

### Quantitative Polymerase Chain Reaction Analysis

To explore further the observation of disparate F. nucleatum read counts between tumour and matched normal samples in our RNA-Seq data set, we developed a targeted quantitative real-time polymerase chain reaction (qPCR) assay to interrogate additional samples. To design the qPCR primers and probe, we gathered the 51,677 read pairs from tumour sample 1 that matched F. nucleatum and performed a local de novo assembly using Short Sequence Assembly by K-mer Search and Extension (SSAKE; Warren et al. 2007) to obtain 861 total contigs, ranging in length from 100 to 1,433 bp.

More specifically, a custom TaqMan primer/probe set was designed to amplify F. nucleatum DNA that matched the contiguous sequence from the WTSS experiment. The cycle threshold (Ct) values for Fusobacterium were normalized to the amount of human biopsy gDNA in each reaction by using a primer/probe set for the reference gene, prostaglandin transporter (PGT), as previously described (Wilson et al. 2006). The reaction efficiency for the Fusobacterium assay and the PGT assay were found to be 97% and 98% respectively. The fold difference (2-DDCt) in Fusobacterium abundance in tumour versus normal tissue was calculated by subtracting DCttumour from DCtnormal where DCt is the difference in threshold cycle number for the test and reference assay. Isolated biopsy DNA was quantified by PicoGreen Assay (invitrogen) on a Wallac Victor spectrophotometer (Perkin Elmer). Each reaction contained 5 ng DNA and was assayed in duplicate in 20 ml reactions containing 1× final concentration TaqMan Universal Master Mix (ABI part number 4304437), 18 mM of each primer and 5 mM probe and took place in a 384-well optical PCR plate. Amplification and detection of DNA was performed with the ABI 7900HT Sequence Detection System (Applied Biosystems) using the reaction conditions: 50° C. for 2 minutes, 95° C. for 10 minutes and 40 cycles of 95° C. for 15 seconds and 60° C. for 1 minute. Cycle thresholding was calculated using the automated settings for SDS 2.2 (Applied Biosystems). Primer and probe sequences for each assay are as follows: Fusobacteria forward primer, 5′CAACCATTACTTTAACTCTACCATGTTCA 3′ (SEQ ID NO: 1); Fusobacteria reverse primer, 5′ GTTGACTTTACAGAAGGAGATTATGTAAAAATC 3′ (SEQ ID NO: 2); Fusobacteria FAM probe, 5′ GTTGACTTTACAGAAGGAGATTATGTAAAAATC 3′ (SEQ ID NO: 3); PGT forward primer, 5′ ATCCCCAAAGCACCTGGT TT 3′ (SEQ ID NO: 4); PGT reverse primer, 5′ AGAGGCCAAGATAGTCCTGGTAA 3′ (SEQ ID NO: 5); PGT FAM probe, 5′ CCATCCATGTCCTCATCTC 3′ (SEQ ID NO: 6). The entire qPCR experiment was performed a second time using the same samples and methods as outlined above, for the purpose of replication, and very similar results were obtained.

The majority of the 861 contigs matched genes encoding F. nucleatum ribosomal RNAs and proteins, but we also obtained 82 contigs that gave BLASTN alignments of 80% or greater sequence identity to other F. nucleatum protein coding genes, as shown in Table 2.

TABLE 2
contig926|size135|read3|cov1.69 ref_NC_003454.1_:c171383-
169923membrane-boundO-acyltransferase[Fusobacteriumnucleatumsubsp.nucleatumATCC25586]
AAGCCTAAAAAAGCTAGTCACAGAAAAAGAGGACATAGACAACTTTTTACTGAAATAAAAGTA
AAACTTCAATTAGCATAGTTATGATTAAGGTAGAAATTTTTAGAAAAAATGGTAGTATAATAGG
ATATAAAG (SEQ ID NO: 7)
contig925|size141|read5|cov2.51 ref_NC_003454.1_:c549922-
549746proteintranslocasesubunitSecE[Fusobacteriumnucleatumsubsp.nucleatumATCC25586]
AAAACTCCAAGATAGATAGATACAAAAACAGTCATGGTTACAACCCATAGAGTAGAATGAATA
ACTTCTGTTTTTGAAGGCCATTCAACTTTTGAGTATTCCATTTTAACCTTTTGAAATAAATTCAT
GCTATCTCCTTTC (SEQ ID NO: 8)
contig923|size78|read2|cov1.69 ref_NC_003454.1_:1725103-1725537neutrophil-
activatingproteinA[Fusobacteriumnucleatumsubsp.nucleatumATCC25586]
AGATTTAAAGCTATACATGAATACACAGAATCATTATATGATTATTATTTTGAAAAATTTGATG
AAGTTGCTGAAGAA (SEQ ID NO: 9)
contig91|size126|read16|cov9.65 ref_NC_003454.1_:1964996-
1965190hypotheticalproteinFN1309[Fusobacteriumnucleatumsubsp.nucleatumATCC25586]
CAAAATTAAGGAGGATAAAAAATGGTTACAGGAGATATGAATATAATGGAAGCAGTTGAAAAA
TACCCAGTAATAATTGAAGTTTTACAAAGAAATGGATTAGGCTGTGTAGGATGCATGATAGCT
(SEQ ID NO: 10)
contig902|size112|read3|cov2.04 ref_NC_003454.1_:c1673874-16730353-hydroxybutyryl-
CoAdehydrogenase[Fusobacteriumnucleatumsubsp.nucleatumATCC25586]
TTTTAATCCTGATCCTATTTCAGTGATTGATAAAGAAGAAGTATTAGTTGCAAATATAGCTTCTG
GTTTACAAATATCATCTAATTCTTTAAAAGTTTGTTTTTTAATTTCC (SEQ ID NO: 11)
contig892|size91|read2|cov1.67 ref_NC_003454.1_:1128717-
1129361Uracilphosphoribosyltransferase[Fusobacteriumnucleatumsubsp.nucleatumATCC25586]
GGAGTTTATAGAAATGAAGAAACTCTTGAGCCTGTTTACTACTATTGTAAACTACCTACTGATA
TAGCTTCAAGAAAGGTTATTCTTGTTG (SEQ ID NO: 12)
contig891|size80|read3|cov2.48 ref_NC_003454.1_:c1942765-1941737DNA-
directedRNApolymerasesubunitalpha[Fusobacteriumnucleatumsubsp.nucleatumATCC25586]
ATTTTCATATCATGAGATTTATCATCAATAACTTGAATGTCCATTGGTTCATCAACCATTTCTTC
TATATCATCTCTTAA (SEQ ID NO: 13)
contig889|size134|read3|cov1.70 ref_NC_003454.1_:441419-
444013ClpBprotein[Fusobacteriumnucleatumsubsp.nucleatumATCC25586]
ATAAAGATGGAAAAGATGATTCTCTTTTAAAACAAGAAGTTACGGCTGATGAAATTGCTGATAT
AGTTTCAAGATGGACAGGTATCCCTGTATCAAAACTTACTGAAACTAAAAAAAAGAAAAAATG
TTACATC (SEQ ID NO: 14)
contig873|size202|read8|cov2.87 ref_NC_003454.1_:484759-485325
AlkylhydroperoxidereductaseC22protein[Fusobacteriumnucleatumsubsp.nucleatumATCC25586]
CACATTTGTGTGTCCAACTGAATTAGAAGATTTACAAGATCACTATGAAGATTTCAAAAAAGAA
GGAGCAGAAGTTTATTCAGTTTCTTGTGACACTGCATTTGTTCACAAAGCATGGGCAGATCATT
CAGAAAGAATTAAAAAAGTTACTTACCCAATGGTAGCTGACCCAACTGGATTCTTAGCAAGAGC
TTTTGAAGTT (SEQ ID NO: 15)
contig844|size86|read2|cov1.77 ref_NC_003454.1_:441419-
444013ClpBprotein[Fusobacteriumnucleatumsubsp.nucleatumATCC25586]
GGAAGAATAGTGGATTTTAAAAATACTTTAATTATAATGACATCTAATATAGGTAGCCATTTAA
TACTTGAAGACCCTGCTCTTTC (SEQ ID NO: 16)
contig970|size85|read2|cov1.79 ref_NC_003454.1_:441419-
444013ClpBprotein[Fusobacteriumnucleatumsubsp.nucleatumATCC25586]
TAAAAAAGAAAAAATGTTACATCTTGAAGACCATATAAAAGAAAGAGTTAAAGGACAAGATGA
AGCTGTTAAAGCTGTTGCAGAC (SEQ ID NO: 17)
contig801|size77|read2|cov1.97 ref_NC_003454.1_:c77526-
76342elongationfactorTu[Fusobacteriumnucleatumsubsp.nucleatumATCC25586]
CATATTCAATGTGAGCTGTATTGATAGTTATTCCTCTTTCTTTTTCTTCAGGAGCAGCATCAATT
TGGTCAAAATCC (SEQ ID NO: 18)
contig792|size100|read4|cov2.90 ref_NC_003454.1_:c349745-
348639majoroutermembraneprotein[Fusobacteriumnucleatumsubsp.nucleatumATCC25586]
ACTTTTTCAGGAGCTGGAGTAGGTGCAGGCATAACTTCCTTAGCTGATGCAACTGATCCAACTA
CTAATAATGAACCTAATACTAATGCTAATTTTTTTC (SEQ ID NO: 19)
contig782|size102|read3|cov1.98 ref_NC_003454.1_:1157570-
1157770hypotheticalproteinFN0507[Fusobacteriumnucleatumsubsp.nucleatumATCC25586]
CTTTGCTAGTAATTTTCATAAATATTCTCCCTTTATTTTTAAATAAAAAAACCGACCTATCGCCT
CATTATGGTTTTAGTCGTCAAACACAACAAGTGGTAG (SEQ ID NO: 20)
contig754|size84|read2|cov1.81 ref_NC_003454.1_:c1832363-1828797pyruvate-
flavodoxinoxidoreductase[Fusobacteriumnucleatumsubsp.nucleatumATCC25586]
TGATAAGTGAGCTACACCAGCTAAGTCCATTACTTCTTGTACTGAATTTGTTGCAAACATTGCA
AACCCAGTTTGTCTTGCTGC (SEQ ID NO: 21)
contig749|size77|read2|cov1.97 ref_NC_003454.1_:1139707-1140915Acetyl-
CoAacetyltransferase[Fusobacteriumnucleatumsubsp.nucleatumATCC25586]
TTGGAAGACAAGTTTCTATAAGAGCTGGAATCCCTTATGAAGTCCCAGCTTATTCTGTAAACAT
AATTTGTGGAAGC (SEQ ID NO: 22)
contig736|size93|read2|cov1.63 ref_NC_003454.1_:c403298-
402825hypotheticalproteinFN1910[Fusobacteriumnucleatumsubsp.nucleatumATCC25586]
CTTCCATTTCTTTTATTACTCTGTCTGTTATATCTTCTCCACCAACTTTTAATGCTTCTGCTTCAA
AAATATAATCATATTTTCCATCTGCTG (SEQ ID NO: 23)
contig721|size133|read3|cov1.71 ref_NC_003454.1_:c1832363-1828797pyruvate-
flavodoxinoxidoreductase[Fusobacteriumnucleatumsubsp.nucleatumATCC25586[
ATTTCTTTTGCAAAATCTGTATTTAATGCTTCTAATTTTGGAACAAGTATATCTCTAATTTCTCTT
GTTCTTACAGAATATTGTCTATTTGTTATCCAATCTTTGAATAAAGTAGCTATATCTTCATTATT
TG (SEQ ID NO: 24)
contig70|size83|read23|cov21.06 ref_NC003454.1_:778593-
778820Acylcarrierprotein[Fusobacteriumnucleatumsubsp.nucleatumATCC25586]
AGAAGAATTTGGAGTAGAAATTCCAGATACAGAAGCAGAAAAAATTAAAACTGTACAAGATGT
TATAAACTATATAGAAGCAC (SEQ ID NO: 25)
contig62|size105|read19|cov13.12 ref_NC_003454.1_:c273354-
272989hypotheticalproteinFN1792[Fusobacteriumnucleatumsubsp.nucleatumATCC25586]
TTTTAATTACTTTTTAAAACTATTTTGCTTCAACAGTTACTGGTGCTTCTTTAAGGTCTCCAAGA
GTTAAAACTCCTTCTGTACCTTTAATAGCAACTTCATTTC (SEQ ID NO: 26)
contig596|size91|read3|cov2.51 ref_NC_003454.1_:c77526-
76342elongationfactorTu[Fusobacteriumnucleatumsubsp.nucleatumATCC25586]
TCCATGGTCAACGTGCCCAATTGTTCCAATGTTTACATGTGGTTTACTTCTTTCATATTTTTCTTT
AGCCATTTTTTCCTCCTAATTAATT (SEQ ID NO: 27)
contig559|size119|read2|cov1.28 ref_NC_003454.1_:1486635-
1487651periplasmiccomponentofeffluxsystem[Fusobacteriumnucleatumsubsp.nucleatumATCC25586]
TTATATGTGTAAGGATTTACTTATACAGGACTCTCACCTTATGCGGTTTATCTTTCCATACAATT
CTAATTCATCAATACACTATATTGAATATCTTACAGTTCTTCACTACTTTGTCC
(SEQ ID NO: 28)
contig555|size100|read3|cov2.28 ref_NC_003454.1_:441419-
444013ClpBprotein[Fusobacteriumnucleatumsubsp.nucleatumATCC25586]
TGGAGGTATTGATGAATATAAGAGGGAATAAAAAAGTAGATAATCAAAACCCAGAAGCAACTT
ATGAAGTTTTAGAAAAATATGCAAAAGATTTAGTTGA (SEQ ID NO: 29)
contig547|size98|read3|cov2.33 ref_NC_003454.1_:1106662-
1107207sigma_54_modulationprotein[Fusobacteriumnucleatumsubsp.nucleatumATCC25586]
ATGCCACTTTAGCTGCTTCTAAATTAAAAACTGGTAATGCACATGTTACAGAAATTTTAGCTTAT
CTAAGTGGAAGCACATTGAAAGCCACTGCAACT (SEQ ID NO: 30)
contig541|size86|read3|cov2.24 ref_NC_003454.1_:c349745-
348639majoroutermembraneprotein[Fusobacteriumnucleatumsubsp.nucleatumATCC25586]
CCTCCATACCATCTGTATTGAACATCAACTGATCCATTTGGTCTCCAAGCAGGAGCAACTTCTCT
GTCTCTGTAAACAATAACTGG (SEQ ID NO: 31)
contig540|size154|read5|cov2.47 ref_NC_003454.1_:c1942765-1941737DNA-
directedRNApolymerasesubunitalpha[Fusobacteriumnucleatumsubsp.nucleatumATCC25586]
AACCTCTATATAAAGGTTCTACAATAAATTGAGATTTGTAATTACTTTCTTTAACTTCTGTTATC
TTTATTGCTTTAGCCTGCTTTTCTATTTTTAACATTATATCAACTCCTATCAAAAGGATTACATTA
TCTTGAATAAAATTCAACTATTA (SEQ ID NO: 32)
contig526|size114|read4|cov2.67 ref_NC_003454.1_:c411670-
410144HDsuperfamilyhydrolase[Fusobacteriumnucleatumsubsp.nucleatumATCC25586]
TCATCATCACTAATCTTATCTGGATTGATTACTATTCTTAATTCTCTACCTGCTTGAATAGCATA
AGAAGATTCTACACCTTCAAATGAGTTTGCTATTTCTTCAAGATTTTCT (SEQ ID NO: 33)
contig517|size136|read4|cov2.24 ref_NC_003454.1_:c77526-
76342elongationfactorTu[Fusobacteriumnucleatumsubsp.nucleatumATCC25586]
CTCTGATTATAGGGATGTCATCTCCTGGGAATCCATATTCATTTAATAATTCTCTAACTTCCATT
TCTACTAATTCTAGTAATTCTTCGTCATCAACCATATCAGATTTGTTTAAGAAAACAACAATATA
TGGAAC (SEQ ID NO: 34)
contig511|size95|read3|cov2.40 ref_NC_003454.1_:c1832363-1828797pyruvate-
flayodoxinoxidoreductase[Fusobacteriumnucleatumsubsp.nucleatumATCC25586]
TAGACCAATTTCTTGTGCAAGGGCAGTAGCATTTATAATAAATAATCTTGCCTTATTTTTAGCTA
AATCTCTTTTTACATTATTTGGTATATTTT (SEQ ID NO: 35)
contig50|size77|read16|cov15.75 ref_NC_003454.1_:1964996-
1965190hypotheticalproteinFN1309[Fusobacteriumnucleatumsubsp.nucleatumATCC25586]
AAGGAATTGAAGCTCACGGATTAGATGCTAAGGCTATTCTTGATGAAATTAATTCTCTAATAAA
AGAATAATAAATT (SEQ ID NO: 36)
contig505|size99|read2|cov1.54 ref_NC_003454.1_:c1942765-1941737DNA-
directedRNApolymerasesubunitalpha[Fusobacteriumnucleatumsubsp.nucleatumATCC25586]
ACCAGAACTTTCAGCCTTTACAACAATTTCTTTAACATTTAAGATAATTTCTGTAACAGCTTCTT
TAACACCATCCATAACAGTAAATTCACTTAAAAC (SEQ ID NO: 37)
contig49|size110|read33|cov22.40 ref_NC_003454.1_:778593-
778820Acylcarrierprotein[Fusobacteriumnucleatumsubsp.nucleatumATCC25586]
GGAAGATATGTTAGATAAAGTAAAAGAAATTATAGTTGAACAATTAGGAGTGGATGCTGATCA
AATAAAACCTGAATCAAATTTTGTAGATGATTTAGGAGCAGATTCTT (SEQ ID NO: 38)
contig472|size84|read2|cov1.81 ref_NC_003454.1_:1139707-1140915Acetyl-
CoAacetyltransferase[Fusobacteriumnucleatumsubsp.nucleatumATCC25586]
GGAGTTTAGCACCTTTAAAACCTGGAGATTTGGGAGCTCAAATAGTAAAAAATATTCTTGAAGA
AACAAAAGTAGATCCAGCTA (SEQ ID NO: 39)
contig469|size100|read2|cov1.39 ref_NC_003454.1_:c411670-
410144HDsuperfamilyhydrolase[Fusobacteriumnucleatumsubsp.nucleatumATCC25586]
GAAAGCTCTATAAGAGCTCCTTCACCAGCAGCAATTATTTCTTTTTCAATTTCTTTTTTACATTT
ATTTACAATTTCTTCTATCTTACCTGGATGTATTC (SEQ ID NO: 40)
contig436|size100|read3|cov2.28 ref_NC_003454.1_:1991809-
1993290transposase[Fusobacteriumnucleatumsubsp.nucleatumATCC25586]
TTTTCCATCTTCAAAAATTTGAGTCATTCCAATTTTCTTTCCTAAAATTCCAGACATTTTTAACCT
CCATCAAATAATATATTGGTTGATACAACTTACC (SEQ ID NO: 41)
contig424|size83|read3|cov2.75 ref_NC_003454.1_:c77526-
76342elongationfactorTu[Fusobacteriumnucleatumsubsp.nucleatumATCC25586]
CTTGTCTTGAAAGTAAGATATGTTCTCTTGTTTGAGGCATAGGTCCATCAGCAGCTGATACAAC
AAGTATAGCTCCGTCCATT (SEQ ID NO: 42)
contig422|size104|read3|cov2.19 ref_NC_003454.1_:c79696-
77615proteintranslationelongationfactorG_EF-G_[Fusobacteriumnucleatumsubsp.nucleatumATCC25586]
GTTAGCTCCAATTCTATCCATCTTGTTAAAGAAAGCTAGTCTTGGTACTTTATATTTATCAGCTT
GTCTCCACACTGTTTCTGATTGTGGTTGTACACCATCAA (SEQ ID NO:43)
contig419|size88|read2|cov1.73 ref_NC_003454.1_:c411670-
410144HDsuperfamilyhydrolase[Fusobacteriumnucleatumsubsp.nucleatumATCC25586]
TTTTACACCATCAAAACAAGAAAGTACAACAGCTTCTGGTGTATCATCTATAATCACATCAACA
CCTGTTAAAGCCTCAATAGTTCTA (SEQ ID NO: 44)
contig414|size137|read6|cov3.05 ref_NC_003454.1_:c132450-
131170preproteintranslocasesubunitSecY[Fusobacteriumnucleatumsubsp.nucleatumATCC25586]
AATGTTGTTTTAATAGATAATGTAGAAGGTAGAGCATTTACTATAACTCCTGGGATTAACATAA
ATACTGAGGCAAAGATTACAGGCATTACTCCTGCTGTGTTAAGTCTCAATGGTATAAATGATTT
TTCTCCTAT (SEQ ID NO: 45)
contig404|size132|read4|cov2.30 ref_NC_003454.1_:c132450-
131170preproteintranslocasesubunitSecY[Fusobacteriumnucleatumsubsp.nucleatumATCC25586]
CCTTTTGAACTAAATCCTTTTCCAACATAATGAATAGGGATTTTTCTTTGTCCAAGTTGAAATAA
TACTATTCCAGCTATTGTTACTGTACCAAGAAATGCTACTAATACCAATAAAGGTATTAAGAAT
TTA (SEQ ID NO: 46)
contig39|size129|read40|cov23.26 ref_NC_003454.1_:1909508-
191019434kDamembraneantigenprecursor[Fusobacteriumnucleatumsubsp.nucleatumATCC25586]
GCAGGTGCAGTTGCTTCTGCAGCAGGTTGTTCAGCTGGTTTTTCTTCTTCTTTCTTTTCTCCACA
AGCTACTAAGAATAAACTCATAGCTAGTGCTAACATTGCAAATTTTTTCATTGTTGATCCCTCC
(SEQ ID NO: 47)
contig391|size200|read16|cov5.99 ref_NC_003454.1_:1785609-
1786040putativecytoplasmicprotein[Fusobacteriumnucleatumsubsp.nucleatumATCC25586]
GCACAATTAGGTTGGATTGCAACAGTAAGAGAAGATGGAGCACCAAACATTGGACCAAAAAGA
TCTTGTCGTATATATGATGATGCAACTTTAATATGGAATGAAAATACAGCTGGTGAAATTATGA
AAGATATTGAAAGAGGTTCAAAAGTTGCAATAGCTTTTGCTAACTGGGATAAGTTAGATGGATA
TCGTTTTGT (SEQ ID NO: 48)
contig380|size129|read2|cov1.17 ref_NC_003454.1_:c79696-
77615proteintranslationelongationfactorG_EF-G_[Fusobacteriumnucleatumsubsp.nucleatumATCC25586]
TGTTATTTTATTAACAAATTCAAATTCTTTACCTGGATTTGGTTCAAGTATAATCTTAACATGTC
CATATTGTCCTCTACCACCAGATTGTTTTGCATACTTAACTTCTTGATCACAAGATTGAGTTAT
(SEQ ID NO: 49)
contig379|size93|read3|cov2.33 ref_NC_003454.1_:c1332180-
133056160kDachaperoninGROEL[Fusobacteriumnucleatumsubsp.nucleatumATCC25586]
TTCCCTTTTCTTCTGATATAACTTCTCCACCAGTTAGAATAGCAATATCTTCAAGTATAGCTTTT
CTTCTATCTCCAAAAGCAAGGAGCTTTA (SEQ ID NO: 50)
contig369|size85|read3|cov2.36 ref_NC_003454.1_:c349745-
348639majoroutermembraneprotein[Fusobacteriumnucleatumsubsp.nucleatumATCC25586]
AAGTTTCTATATTCAGCACCAGCAGCTGCATATAATTTTACGAAATCTGTTGGTTTATAAGAAA
CTTGGAAAGTTGGTAACATAT (SEQ ID NO: 51)
contig356|size79|read2|cov1.92 ref_NC_003454.1_:1782271-
1783119phosphonates-bindingprotein[Fusobacteriumnucleatumsubsp.nucleatumATCC25586]
CTTTTGCATATATTTTAGCAAATAAAAAGAATGGAACAGAAGCATTACTTACAAGTATAAATAA
ACATGATGAACCTGG (SEQ ID NO: 52)
contig34|size448|read143|cov23.10 ref_NC_003454.1_:891002-
891391hypotheticalproteinFN0264[Fusobacteriumnucleatumsubsp.nucleatuniATCC2SSS6]
ATTTATTATAATTTAATTTGGGAGGTAACAAAAATGAAAAAATTTTTATTATTAGCAGTATTAG
CTGTTTCTGCTTCAGCATTTGCAGCAAATGATGCAGCAAGTTTAGTAGGTGAATTACAAGCATT
AGATGCTGAATACCAAAACTTAGCAAATCAAGAAGAAGCAAGATTTAATGAAGAAAAAGCACA
AGCTGATGCCGCTAAACAAGCACTAGCACAAAATGAACAAGTTTACAATGAATTATCTCAAAG
AGCTCAAAGACTTCAAGCTGAAGCTAACACAAGATTTTATAAATCTCAATATCAAGAATTAGCT
TCTAAATATGAAGATGCTTTAAAGAAATTAGAAGCTGAAATGGAACAACAAAAAGGTGTCATT
TCTGACTTTGAAAAGATTCAAGCTTTAAGAGCTGGTAACTAATAAATTTTGAAAAAATGCTAGC
ATG (SEQ ID NO: 53)
contig339|size94|read2|cov1.62 ref_NC_003454.1_:c326804-323994TPRrepeat-
containingprotein[Fusobacteriumnucleatumsubsp.nucleatumATCC25586]
TAATACTTTTTGAAAGTCTGCTTCTGCTTCATCATATTTTCCAAGTCCCATAGCAGCTATACCTT
TTAAATAGTTCACACTGCTTTCATCTTGT (SEQ ID NO: 54)
contig309|size109|read2|cov1.39 ref_NC_003454.1_:c886099-884255Zinc-
transportingATPase[Fusobacteriumnucleatumsubsp.nucleatumATCC25586]
ATTGCTAAACCTATAACTATTGGTGTATATATTTTAGAAAATCTTGTTATTAATCTTTCAGATTT
TGACTTTTTAGCTGAGGCATTTTCTACTAAATCTAAAACTTTAT (SEQ ID NO: 55)
contig307|size77|read2|cov1.97 ref_NC_003454.1_:c1678570-1678262DNA-
bindingproteinHU[Fusobacteriumnucleatumsubsp.nucleatumATCC25586]
TTTTTAACTTCTCTCGCACTTCTTTCTTTTACTTCCCAAGAACCAAATCCTATGAATCTTACTCCA
TCACCTTTTAG (SEQ ID NO: 56)
contig301|size88|read3|cov2.30 ref_NC_003454.1_:c1118255-
1117752flavodoxinFldA[Fusobacteriumnucleatumsubsp.nucleatumATCC25586]
CCATCATCTTGTAATTCTCCTGCACCATAAGTTGGAGAAGCCATTATAATGTTATCAAAAACTTC
CATTTCAGCAACACCATTAGCAA (SEQ ID NO: 57)
contig294|size244|read12|cov3.74 ref_NC_003454.1_:1760699-
1761028hypotheticalproteinFN1118[Fusobacteriumnucleatumsubsp.nucleatumATCC25586]
ACTGAAATAAAAGTAACTTCAATAACCATAGTTATGATTAAGGTAGAAATTTTTAGAAAAAATG
GTAGTATAATAGGATATAAAGCAAATGGACATTCTGGATATTCAGAACAAGGTAGTGATATCAT
CTGTTCTGCTATCTCAACATCATTACAAATGACTTTGGCAGGAATTCAAGAAGTGTTAAAGTTA
GAACCTAAATTTAAAATGAATGATGGTTTTCTTGATGTTGATTTAAGAAATA
(SEQ ID NO: 58)
contig293|size121|read6|cov3.77 ref_NC_003454.1_:441419-
444013ClpBprotein[Fusobacteriumnucleatumsubsp.nucleatumATCC25586]
GTTGTTAGAATAGATATGAGTGAATATATGGATAAGTTTTCAGTTACAAGACTTATAGGTGCAC
CTCCAGGATATGTTGGTTATGAAGAAGGAGGACAACTTACAGAAGCTATTAGAACTA
(SEQ ID NO: 59)
contig276|size248|read13|cov3.98 ref_NC_003454.1_:c1118255-
1117752flavodoxinFldA[Fusobacteriumnucleatumsubsp.nucleatumATCC25586]
TATCATATGAAGCATCTATTGCAAGTCCCATAAATTTATCTCCATCAATAACTGCTTCAGAAGCT
TCAAAATCGTATCCATCAGTAGAAGTAAATCCAACTATTTTAGCTCCTTTAGGTTGAACTGCATC
ATATAAATGTTTCAAAGCTTCAACATAGTTTCCACCAAATATTGCAGCATCTCCTACACCAACTA
ATGCAACAACTTTTCCAGAGAAGTCCATATCAGCAACTTCATCAATAACAGAA (SEQ ID NO: 60)
contig269|size82|read3|cov2.78 ref_NC_003454.1_:441419-
444013ClpBprotein[Fusobacteriumnucleatumsubsp.nucleatumATCC25586]
GATGAAATTGAAAAAGCTCACCCTGATGTATTTAATGTGCTATTACAAGTTTTAGATGATGGTA
GACTTACAGATGGACAAG (SEQ ID NO: 61)
contig267|size100|read4|cov3.04 ref_NC_003454.1_:c1832363-1828797pyruvate-
flavodoxinoxidoreductase[Fusobacteriumnucleatumsubsp.nucleatumATCC25586]
TAAATATTGGCCAATATCCACATTCAGTAGCAAGCTTCATTTCAGTTTGAGATTTTGACATACCT
TTCTTAATACCATGGTTGATACAAGGTGAGTATGC (SEQ ID NO: 62)
contig266|size100|read3|cov2.28 ref_NC_003454.1_:c132450-
131170preproteintranslocasesubunitSecY[Fusobacteriumnucleatumsubsp.nucleatumATCC25586]
TGAGGAATAATAGATACAAGCAAACTTACAACTATTGAAGCATTGATGTAAGGAATAATTCCTA
ATGAGAATATAGATATTCTTGTAAAAGCTCCACCAG (SEQ ID NO: 63)
contig262|size161|read6|cov2.83 ref_NC_003454.1_:c549749-
549168transcriptionantiterminationproteinnusG[Fusobacteriumnucleatumsubsp.nucleatumATCC25586]
TCCAAAAATATCAACCATTACTTTAACTCTACCATGTTCATGATCTATTTCAGCAACTTGTCCTT
CTTGATCTTTAAATGAACCTTTTAAGATTTTTACATAATCTCCTTCTGTAAAGTCAACTTTTATA
GTTTCTTTAGGTGTCTTTACACCTATTATAT (SEQ ID NO: 64)
contig261|size240|read17|cov5.38 ref_NC_003454.1_:c1832363-1828797pyruvate-
flavodoxinoxidoreductase[Fusobacteriumnucleatumsubsp.nucleatumATCC25586]
AAGGTGAGTATGCAATAATTATTGATGGTCCTTGGTGAGCTTCTGCTTCCTTAACAGCCTTAAT
AAATTGTTGTTGATTAGCTCCCATAGAAACTTGTGCTACATAGATATGTCCATAAGACATTGCT
ATTGCAGCTAAATCTTTTTTCTTAACTGGTTTTCCAGCTGCTGCAAATTTTGCAACTGCTCCAGT
AGGTGTAGCTTTTGATGCTTGTCCACCAGTATTTGAATAAACTTCTG
(SEQ ID NO: 65)
contig248|size100|read6|cov4.13 ref_NC_003454.1_:c1942765-1941737DNA-
directedRNApolymerasesubunitalpha[Fusobacteriumnucleatumsubsp.nucleatumATCC25586]
TAGAATTTCCTCCTCTTTTCAAAATTATTCAGGAGATCCATTTTTTTCAAGATCATATCCTAAAT
CTTTCATCTTTTCTAAAATCTCATCTAAAGATTTC (SEQ ID NO: 66)
contig241|size123|read10|cov6.18 ref_NC_003454.1_:729309-
729620thioredoxin[Fusobacteriumnucleatumsubsp.nucleatumATCC25586]
AATTGGTGTGGGCCTTGTAAAAGTTTAGTGCCTATACTTGAGGAAGTTGTTGAAGAAGATCCAA
GTAAAAAAATAGTAAAAGTAGATATAGATGAACAAGAAGAACTAGCAACACAATATAAA (SEQ
ID NO: 67)
contig227|size86|read3|cov2.65 ref_NC_003454.1_:c1118255-
1117752flavodoxinFldA[Fusobacteriumnucleatumsubsp.nucleatumATCC25586]
ATTTAGCAGCTATGATATCAGCAACTTCTTGAGTTTTTCCTCCTGTAGTTCCAAAAAATATACCA
ACTGTTTTCATTTAAAATTAC (SEQ ID NO: 68)
contig221|size83|read5|cov4.58 ref_NC_003454.1_:1785609-
1786040putativecytoplasmicprotein[Fusobacteriumnucleatumsubsp.nucleatumATCC25586]
TCGTTTTGTAGGGACAGCAGAAGTTCATAAAGAAGGAAAATATTATGATGAAGCTGTTGAATG
GGCAAAAGGAAAAATGGGAG (SEQ ID NO: 69)
contig202|size83|read3|cov2.75 ref_NC_003454.1:c132450-
131170preproteintranslocasesubunitSecY[Fusobacteriumnucleatumsubsp.nucleatumATCC25586]
AAATACTGTTCCAGCTGTTAGGGTTGTTATTGTTCTTACAAAAAAACTTATTCCTGGATTATAGA
TCAAACCAACAGATTGTA (SEQ ID NO: 70)
contig190|size193|read8|cov3.15 ref_NC_003454.1_:c77526-
76342elongationfactorTu[Fusobacteriumnucleatumsubsp.nucleatumATCC25586]
AACTCTTCCTGTAACAACTGTTCCTCTTCCTGTGATAGTGAAAACATCTTCTATTGGCATTAAGA
ATGGTTGATCTATTGCTCTTTCTGGAGTAGGGATATAGTTATCTACTGCTTCCATAAGTTCTAAT
ATTTTTTCAACCCATTTTTCTTCACCATTTAAAGCACCTAATGCTGAACCTCTGATTATAGGG
(SEQ ID NO: 71)
contig156|size186|read11|cov4.49 ref_NC_003454.1_:c77526-
76342elongationfactorTu[Fusobacteriumnucleatumsubsp.nucleatumATCC25586]
TTCCTCTTAATAACACTCCAATGTTATCTCCTGCTTGACCTTGATCAAGAAGTTTTCTAAACATT
TCAACACCTGTACAAGTTGTTTTAGTTGTAGGTTTGATACCAACTATTTCAATTTCTTCTCCAAC
TTTGATAACTCCTCTTTCAACTCTTCCTGTAACAACTGTTCCTCTTCCTGTGATAG
(SEQ ID NO: 72)
contig152|size87|read3|cov2.62 ref_NC_003454.1_:c107268-105943nitrogenfixationiron-
sulphurproteinRNFC[Fusobacteriumnucleatumsubsp.nucleatumATCC25586]
AATTTTTCAATTGGTAAATGTTCTGTTTGTATTTTATTTTCAGGGGGATGGACTCCACCTCTGAA
ACCAAAAAATGTCATTTAAAAC (SEQ ID NO: 73)
contig142|size88|read9|cov7.62 ref_NC_003454.1_:1785609-
1786040putativecytoplasmicprotein[Fusobacteriumnucleatumsubsp.nucleatumATCC25586]
CAAGAATAAGGAGTGATAATAATGGCTAAATTAACAGATGCTATAAAAGATTTAATATTAAAC
CCAGTTAAAGAAGGAGCTTGGACAG (SEQ ID NO: 74)
contig136|size100|read13|cov8.63 ref_NC_003454.1_:c1944753-
1944505proteintranslationinitiationfactor1[Fusobacteriumnucleatumsubsp.nucleatumATCC25586]
TATGCCCATTTTCTAATTCTACTTTAAACATAGCATTAGGTAAGGCTTCTACTATAACACCTTCC
AATTCGATAACATCTTTCTTTGACATTTTTCCTCC (SEQ ID NO: 75)
contig135|size177|read58|cov23.62 ref_NC_003454.1_:c273354-
272989hypotheticalproteinFN1792[Fusobacteriumnucleatumsubsp.nucleatumATCC25586]
TTCTCCAGATGCAGTTTCAGCATTTTTTAATTCAGTTGCTTTTCCATCTGCATCAGTTAAAGTTG
CAGTACTTCCATCAGCAGCAACTACTAATGTAAATTCCTTTCCATCTTCAGTTTTAAGTGAGAAT
GTTTTAGCTTCAGCAGCAGCTTCAGTTGCTGGTGCTTCTGTAGCAGG (SEQ ID NO: 76)
contig119|size98|read8|cov6.20 ref_NC_003454.1_:729309-
729620thioredoxin[Fusobacteriumnucleatumsubsp.nucleatumATCC25586]
AAATAAAAATTTTAGGAGGATTGTAAGATGGCAATTATAAAAGGAACAAAAGAAAATTTTGAA
GCAGAAGTATTAAAAGCAAATGGAGTTGTAGTAGT (SEQ ID NO: 77)
contig1146|size79|read2|cov1.54 ref_NC_003454.1_:595848-
596567permease[Fusobacteriumnucleatumsubsp.nucleatumATCC25586]
TAACATATCAATAATACTTTTGTCAAGAAAAATTATAGTGCAGACTTAGCAGCTTCTACTAATTT
TGTAAATTCAGTAG (SEQ ID NO: 78)
contig1134|size104|read2|cov1.46 ref_NC_003454.1_:c349745-
348639majoroutermembraneprotein[Fusobacteriumnucleatumsubsp.nucleatumATCC25586]
TACTACTTTTTCAGGAGCTGGAGTAGGTGCAGGCATAACTTCCTTAGCTGATGCAACTGATCCA
ACTACTAATAATGAACCTAATACTAATGCTAATTTTTTCA (SEQ ID NO: 79)
contig1110|size92|read2|cov1.65 ref_NC_003454.1_:441419-
444013ClpBprotein[Fusobacteriumnucleatumsubsp.nucleatumATCC25586]
ATCCTAATAGACCTATGGGTTCATTTATATTCTTAGGACCTACTGGTGTTGGTAAGACATACCTT
GCAAAAACTTTGGCATATAACCTATTT (SEQ ID NO: 80)
contig1107|size127|read2|cov1.18 ref_NC_003454.1_:c79696-
77615protemtranslationelongationfactorG_EF-G_[Fusobacteriumnucleatumsubsp.nucleatumATCC25586]
CTCTTCTTGAATTTAAGTCTCCAATAATATCTCCCATATATTCTTCTGGTGTTGTTACTTCAACTT
TGAATACTGGTTCTAATATCACTGGTTTAGCCTTTGCAGCAGCTTGTTTAACAGCCATTGA (SEQ
ID NO: 81)
contig1096|size84|read4|cov2.99 ref_NC_003454.1_:441419-
444013ClpBprotein[Fusobacteriumnucleatumsubsp.nucleatumATCC25586]
TATTAGTAAATGAACCTAATATTGATGATACTATTTCAATATTAAGAGGTCTTAAAGATAAATT
TGAAACTTATCATGGTGTTA (SEQ ID NO: 82)
contig1051|size89|read2|cov1.28 ref_NC_003454.1_:c79696-
77615proteintranslationelongationfactorG_EF-G_[Fusobacteriumnucleatumsubsp.nucleatumATCC25586]
AACCAATGGATATCCAGCAATAACTCCTGATTCAAGAGCTTCTCTACATCCTTTTTCAACAGCA
GGTATATATTCTCTTGGAATTACCC (SEQ ID NO: 83)
contig102|size81|read7|cov6.57 ref_NC_003454.1_:c1309231-
1307807Na+/H+antiporterNHAC[Fusobacteriumnucleatumsubsp.nucleatumATCC25586]
ATAGCTCATTTTGCTATCTTTAATTTAACACCAATTTTATGGTTGCTTTTTGTCTTTTCTCTATTC
TGTTGCTAATGTCCT (SEQ ID NO: 84)
contig1024|size81|read2|cov1.80 ref_NC_003454.1_:c1332180-
133056160kDachaperoninGROEL[Fusobacteriumnucleatumsubsp.nucleatumATCC25586]
TTTATAACAAGAGTTGTTAGAGCTTCTCCTTCAATATCATCAGCTACAATTAAAACTGGCTTAGA
CATTTGCACAGTTTTT (SEQ ID NO: 85)
contig1022|size80|read3|cov2.85 ref_NC_003454.1_:c349745-
348639majoroutermembraneprotein[Fusobacteriumnucleatumsubsp.nucleatumATCC25586]
AGTTTTCAGTTTCTCCATACCATCTGTATTGAACATCAACTGATCCATTTGGTCTCCAAGCAGGA
GCAACTTCTCTGTCT (SEQ ID NO: 86)
contig1016|size100|read2|cov1.52 ref_NC_003454.1_:829712-831466glutaconyl-
CoAdecarboxylaseAsubunit[Fusobacteriumnucleatumsubsp.nucleatumATCC25586]
AGAAATTACTATTAGAAAAGGTTCAGCAGCGGCTCACTATGTATTGGGTGGACCACAAGGTAAT
AATACAAATGCTTTCTCATTAGGAACAGCAGCAACA (SEQ ID NO: 87)
contig1012|size98|read3|cov2.33 ref_NC_003454.1_:c77526-
76342elongationfactorTu[Fusobacteriumnucleatumsubsp.nucleatumATCC25586]
AATACATAAACTTCACCTTTGAAGTTTGTATGAGGGTGGATACTTCCTGGTTTAGCAAGAACTT
GTCCTCTTTCAACTTCTTCTTTCTTAGTTCCTCT (SEQ ID NO: 88)

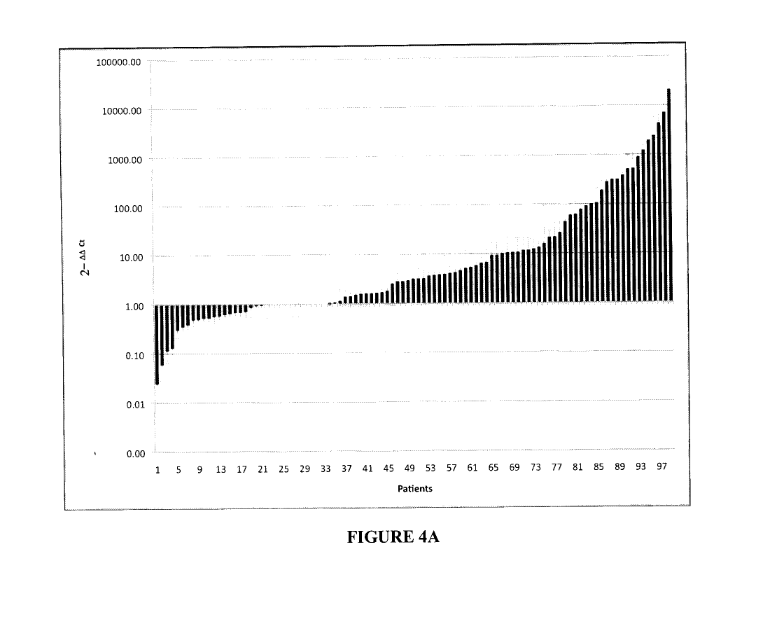

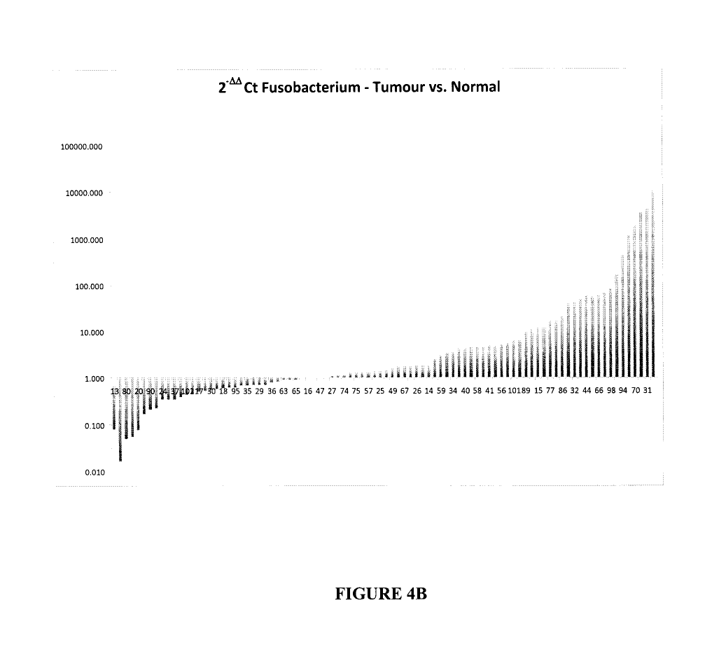

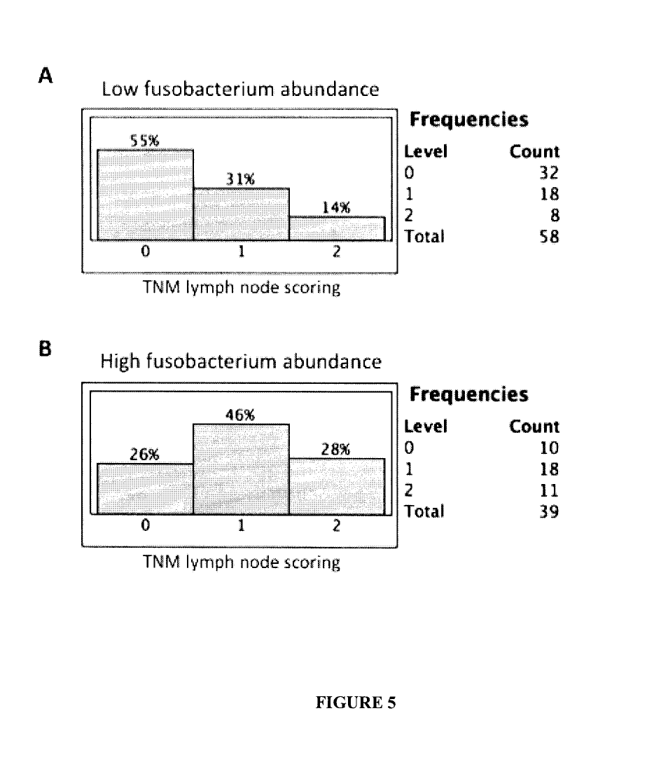

A 161 bp contig that returned a high quality BLAST match (95% identity) to the nusG gene (GenBank accession AAL94126.1) of F. nucleatum, and no match to any gene of any other species, was used as the target for designing a qPCR (Taqman, ABI) primer/probe set. The initial metagenomics screen described above involved interrogation of expressed genes, however, once we established F. nucleatum as a candidate pathogen, we switched to analysis of gDNA because a larger amount of high quality DNA than RNA was obtainable from the frozen tissue sections. We conducted qPCR on gDNA isolated from an additional 88 colorectal carcinomas and matched normal specimens and confirmed an overrepresentation of F. nucleatum in tumour versus matched normal specimens (p=2.5E-6, two-tailed ratio t-test) (FIG. 4A). The Fusobacterium abundance measured by qPCR correlated with that measured from the RNA-seq data (Pearson's r=0.97). The mean overall abundance of Fusobacterium found to be 415 times greater in the tumour samples (n=99) than in the matched normal samples (n=99) (FIG. 4A). A similar validation study using matched and normal pairs of specimens from colorectal carcinoma subjects showed a strong over-representation of the F. nucleatum homologous sequence in tumour compared to matched normal control tissue (p=1.5E-5, ratio t test) (FIG. 4B).

### Example 3

### Isolation, Culture and Whole Genome Sequencing of a Representative Strain of F. nucleatum

We attempted to culture Fusobacteria anaerobically, directly from twelve of the frozen tumour sections that showed high abundance by qPCR, and obtained a single isolate (CC53).

More specifically, frozen tumour sections were thawed and immediately placed into 500 ml of pre-reduced phosphate buffered saline, and the tissue agitated and gently broken up using a pipette fitted with a sterile, wide-bore, plugged tip. 100 ml aliquots of this suspension were directly spread onto pre-reduced fastidious anaerobe agar (FAA) plates supplemented with 5% defibrinated sheep blood (DSB), and incubated for 10 days in a humidified anaerobe chamber (Ruskinn Bug Box). Plates were inspected every 2 days for growth, and all colonies were picked and streak-purified on further pre-reduced FAA+5% DSB plates. Single colonies were examined by phase microscopy using a Leica ICC50 microscope fitted with a 100× oil immersion objective, looking for slender rods or needle-shaped cells characteristic of F. nucleatum. gDNA was isolated from positively identified isolates using a Maxwell 16 instrument with cell DNA cartridges, and aliquots used as template in PCR with primers and conditions as described by Kim et al. (2004). A product size of 495 nt confirmed that the isolate belonged to the Fusobacterium genus, and a further PCR to partially amplify 16S rRNA gene was carried out using the same DNA template using primers and conditions as defined by Ben-Dov et al. (Ben-Dov et al. 2006). This product was sent for Sanger sequence analysis to MWG Operon, and obtained traces confirmed F. nucleatum as the species. In total, 3 clones of the isolated strain were obtained from the tumour specimen from patient number 53, and named CC53 F, G and H respectively. All strains were stored at −80° C. in cryoprotectant media (12% w/v skim milk powder, 1% (v/v) dimethyl sulfoxide and 1% (v/v) glycerol).

We purified high molecular weight (HMW) gDNA from CC53 culture and constructed and sequenced a whole genome shotgun (WGS) library using the Illumina HiSeq platform.

More specifically, Fusobacterium genomic DNA was sonicated and size fractions between 175 to 200 bp and 400 to 450 bp were isolated following PAGE. WGSS Paired-end Illumina libraries were prepared from each size fraction as described previously with the following modifications: the final PCR amplification was increased to 15 cycles and contained the standard Illumina PE1 PCR primer and an indexed PE2 primer as detailed above for RNA-Seq library construction. A total of 92.0M paired 100 nt reads were obtained from a single lane of the Illumina HiSeq instrument. After quality filtering, keeping only pairs with an average base quality of Q30 or higher, 64.8M paired reads were aligned with novoalign (www.novocraft.com; -o SAM-r A-R 0) onto the F. nucleatum subsp. nucleatum ATCC 25586 (GenBank accession NC_{—}003454.1) and Fusobacterium sp. 3_{—}1_{—}36A2 genome sequences (HMP accessions GG698790-GG698801), respectively. Paired read alignments were processed using custom PERL scripts that tracked genome sequence coverage, depth of coverage and average sequence identity of mapped pairs. Annotation of strain sp. 3_{—}1_{—}36A2 regions devoid of read alignments was performed by extracting the coordinates of alignment gaps 1 kbp or larger and mining the HMP GenBank-format file for existing gene annotations (http://www.hmpdacc.org/data_genomes.php). Reads that did not align onto the sp. 3_{—}1_{—}36A2 genome assembly were quality-trimmed to only include those having 70 or more consecutive Q30 bases and assembled with SSAKE (v3.7-p 1-m 20-o 2-r 0.7) in 67 contigs (mean size=1,225 bp max size=6,018 bp total bases=82,076 bp N50=1,359 bp). The contigs were annotated using BLASTX (v2.2.25), reporting the best hit for each high-scoring pairs and manually inspecting each alignment.

In a separate analysis, the 64.8M paired QC reads were filtered further, leaving only sequences having 99 or 100 consecutive Q30 bases. This aggressive filter yielded approximately 32M total reads, including 4.5M paired and 22.9M unpaired reads and assembled with SSAKE (v3.7-p 1-m 20-o 2-r 0.7) into 379 contigs (mean size=5,460 bp max size=31,878 bp total bases=2,069,558 bp N50=8,680 bp). The Fusobacterium sp. 3_{—}1_{—}36A2 genome assembly was aligned onto the type strain using cross_match (www.phrap.org; -minmatch 29-minscore 59-masklevel 101) and ordered/oriented based on the latter. Fusobacterium tumour isolate contigs were in turn aligned onto the reordered Fusobacterium sp. 3_{—}1_{—}36A2 HMP genome assembly and ordered/oriented according to that genome sequence, using the same cross_match parameters. Three-way cross_match alignments between the ordered Fusobacterium genomes were performed and plotted using hive plots (www.hiveplot.com).

We obtained an excessive number (64,819,156) of quality filtered paired 100 nt reads. These reads were aligned to the F. nucleatum type strain American Type Culture Collection (ATCC) 25586 (Genbank accession NC_{—}003454.1) sequence, covering 76% of this reference genome with 2,661-fold mean depth and 95.6+/−2.0% (mean+/−SD) identity. Further, we aligned reads from CC53 to 483 additional draft genome sequences available from the Human Microbiome Project (HMP) (Nelson et al. 2010) including sixteen as of yet incomplete Fusobacterium genomes. CC53 aligned with highest identity to Fusobacterium sp. 3_{—}1_{—}36A2, covering 91.6% of the 12-supercontig draft assembly with 99.5+/−1.2% (mean+/−SD) sequence identity. Three-way analysis among these strains using cross-match Smith-Waterman alignments confirmed that CC53 is closest to Fusobacterium sp. 3_{—}1_{—}36A2.

More specifically, approximately 32M high-quality WGS Illumina HiSeq reads (>=99 consecutive Q30 bases) from Fusobacterium tumour isolate CC53 were assembled with SSAKE (v3.7, default options) into 379 contigs. The contigs were aligned using cross_match (-minmatch 29-minscore 59-masklevel 101) to the complete F. nucleatum subsp. nucleatum ATCC 25586 genome and, independently to the 12-contig HMP Fusobacterium sp. 3_{—}1_{—}36A2 assembly, respectively and ordered/oriented based on the highest identity to the latter sequence. Three-way cross_match (www.phrap.org) alignments between each Fusobacterium genomes were performed and represented visually using hive plots (www.hiveplot.com). Sequence similarity and synteny was highest between CC53 and sp. 3_{—}1_{—}36A2, as evidenced by a greater density of high similarity sequence matches between them, relative to relative to ATCC 25586, and shared patterns of inversions compared to this reference strain. Three regions of sequences present in sp. 3_{—}1_{—}36A2 but absent from CC53 were apparent as conspicuous gaps on the sp. 3_{—}1_{—}36A2 axis.

Some notable differences were apparent, however. We observed 19 segments from strain 3_{—1}_{—}36A2 that were missing from CC53. The majority (156/206) of the predicted coding sequences (CDS) on these segments from strain 3_{—}1_{—}36A2 had unknown function, but there were numerous sequences indicative of prophage content, including genes encoding putative helicase, integrase, recombinase, terminase and topoisomerase activity (Table 3).

TABLE 3
Gene content of gaps (segments from strain 3_1_36A2 accession that are missing from CC53).

| Coordinates | Unaligned | | | | |
|---|---|---|---|---|---|
| of unaligned | region | | CDS start | CDS end | |
| region | length | Protein id | coordinate | coordinate | Predicted product |
| 160980-162119 | 1139 | EEU32070.1 | 161007 | 162053 | predicted protein |
| 163749-165149 | 1400 | EEU32072.1 | 162670 | 163818 | 2-nitropropane dioxygenase |
| 163749-165149 | 1400 | EEU32073.1 | 163997 | 164458 | N-acetylmuramoyl-L-alanine amidase |
| 163749-165149 | 1400 | EEU32074.1 | 164490 | 165128 | conserved hypothetical protein |
| 163749-165149 | 1400 | EEU32075.1 | 166472 | 165135 | conserved hypothetical protein |
| 230196-237851 | 7655 | EEU32133.1 | 230444 | 234547 | CRISPR-associated protein |
| 230196-237851 | 7655 | EEU32134.1 | 234572 | 235450 | CRISPR-associated protein cas1 |
| 230196-237851 | 7655 | EEU32135.1 | 235440 | 235760 | conserved hypothetical protein |
| 230196-237851 | 7655 | EEU32136.1 | 235757 | 236419 | conserved hypothetical protein |
| 230196-237851 | 7655 | EEU32137.1 | 238229 | 237840 | conserved hypothetical protein |
| 413437-423361 | 9924 | EEU32297.1 | 415519 | 413525 | hemin receptor |
| 413437-423361 | 9924 | EEU32298.1 | 415901 | 416818 | nickel ABC transporter |
| 413437-423361 | 9924 | EEU32299.1 | 416839 | 417642 | nickel ABC transporter |
| 413437-423361 | 9924 | EEU32300.1 | 417655 | 418416 | nickel import ATP-binding protein |
| | | | | | NikD |
| 413437-423361 | 9924 | EEU32301.1 | 418421 | 419113 | nickel import ATP-binding protein |
| | | | | | NikE |
| 413437-423361 | 9924 | EEU32302.1 | 419110 | 420735 | nickel import ATP-binding protein |
| | | | | | NikE |
| 413437-423361 | 9924 | EEU32303.1 | 421767 | 420790 | transcriptional regulator AraC family |
| 413437-423361 | 9924 | EEU32304.1 | 421979 | 423325 | MATE efflux family protein |
| 800252-803854 | 3602 | EEU32662.1 | 800383 | 798980 | MATE efflux family protein |
| 800252-803854 | 3602 | EEU32663.1 | 801168 | 800395 | phosphonate C-P lyase system |
| | | | | | protein PhnK |
| 800252-803854 | 3602 | EEU32664.1 | 802198 | 801161 | transport system permease |
| 800252-803854 | 3602 | EEU32665.1 | 803435 | 802293 | periplasmic binding protein |
| 800252-803854 | 3602 | EEU32666.1 | 803816 | 803643 | predicted protein |
| 804906-806958 | 2052 | EEU32668.1 | 806372 | 805035 | conserved hypothetical protein |
| 804906-806958 | 2052 | EEU32669.1 | 806844 | 806386 | conserved hypothetical protein |
| 854976-876729 | 21753 | EEU32722.1 | 855099 | 855521 | predicted protein |
| 854976-876729 | 21753 | EEU32723.1 | 855650 | 856117 | predicted protein |
| 854976-876729 | 21753 | EEU32724.1 | 860232 | 856126 | predicted protein |
| 854976-876729 | 21753 | EEU32725.1 | 862070 | 860229 | predicted protein |
| 854976-876729 | 21753 | EEU32726.1 | 862942 | 862052 | predicted protein |
| 854976-876729 | 21753 | EEU32727.1 | 864868 | 862955 | predicted protein |
| 854976-876729 | 21753 | EEU32728.1 | 865392 | 864868 | predicted protein |
| 854976-876729 | 21753 | EEU32729.1 | 865593 | 865405 | conserved hypothetical protein |
| 854976-876729 | 21753 | EEU32730.1 | 866029 | 865598 | conserved hypothetical protein |
| 854976-876729 | 21753 | EEU32731.1 | 866417 | 866037 | conserved hypothetical protein |
| 854976-876729 | 21753 | EEU32732.1 | 866976 | 866473 | predicted protein |
| 854976-876729 | 21753 | EEU32733.1 | 867481 | 866978 | predicted protein |
| 854976-876729 | 21753 | EEU32734.1 | 868738 | 867494 | predicted protein |
| 854976-876729 | 21753 | EEU32735.1 | 869642 | 868791 | predicted protein |
| 854976-876729 | 21753 | EEU32736.1 | 870369 | 869653 | predicted protein |
| 854976-876729 | 21753 | EEU32737.1 | 870587 | 870369 | predicted protein |
| 854976-876729 | 21753 | EEU32738.1 | 872229 | 870577 | predicted protein |
| 854976-876729 | 21753 | EEU32739.1 | 872706 | 872239 | predicted protein |
| 854976-876729 | 21753 | EEU32740.1 | 874108 | 872717 | predicted protein |
| 854976-876729 | 21753 | EEU32741.1 | 874781 | 874083 | predicted protein |
| 854976-876729 | 21753 | EEU32742.1 | 875192 | 875425 | predicted protein |
| 854976-876729 | 21753 | EEU32743.1 | 875441 | 875674 | predicted protein |
| 854976-876729 | 21753 | EEU32744.1 | 875664 | 875915 | predicted protein |
| 854976-876729 | 21753 | EEU32745.1 | 876091 | 875912 | predicted protein |
| 854976-876729 | 21753 | EEU32746.1 | 876444 | 876091 | predicted protein |
| 854976-876729 | 21753 | EEU32747.1 | 876724 | 876449 | predicted protein |
| 877026-882644 | 5618 | EEU32748.1 | 877946 | 877146 | replicative DNA helicase |
| 877026-882644 | 5618 | EEU32749.1 | 878688 | 877948 | conserved hypothetical protein |
| 877026-882644 | 5618 | EEU32750.1 | 880025 | 879648 | predicted protein |
| 877026-882644 | 5618 | EEU32751.1 | 880385 | 880164 | predicted protein |
| 877026-882644 | 5618 | EEU32752.1 | 880941 | 880543 | predicted protein |
| 877026-882644 | 5618 | EEU32753.1 | 881131 | 880952 | predicted protein |
| 877026-882644 | 5618 | EEU32754.1 | 881328 | 881131 | conserved hypothetical protein |
| 877026-882644 | 5618 | EEU32755.1 | 882003 | 881347 | LexA repressor |
| 877026-882644 | 5618 | EEU32756.1 | 882166 | 882540 | predicted protein |
| 883266-886895 | 3629 | EEU32757.1 | 883275 | 883967 | conserved hypothetical protein |
| 883266-886895 | 3629 | EEU32758.1 | 883977 | 884489 | gp157 |
| 883266-886895 | 3629 | EEU32759.1 | 884500 | 885222 | predicted protein |
| 883266-886895 | 3629 | EEU32760.1 | 885219 | 885518 | conserved hypothetical protein |
| 883266-886895 | 3629 | EEU32761.1 | 885515 | 885745 | phage protein |
| 883266-886895 | 3629 | EEU32762.1 | 885708 | 886172 | phage protein |
| 883266-886895 | 3629 | EEU32763.1 | 886162 | 886734 | conserved hypothetical protein |
| 883266-886895 | 3629 | EEU32764.1 | 886721 | 886873 | predicted protein |
| 887195-890120 | 2925 | EEU32765.1 | 887406 | 887561 | predicted protein |
| 887195-890120 | 2925 | EEU32766.1 | 887576 | 887806 | predicted protein |
| 887195-890120 | 2925 | EEU32767.1 | 887775 | 888182 | conserved hypothetical protein |
| 887195-890120 | 2925 | EEU32768.1 | 888187 | 888717 | conserved hypothetical protein |
| 887195-890120 | 2925 | EEU32769.1 | 888731 | 888922 | predicted protein |
| 887195-890120 | 2925 | EEU32770.1 | 888960 | 890027 | DNA |
| | | | | | integration/recombination/invertion |
| | | | | | protein |
| 965306-1019450 | 54144 | EEU32841.1 | 966788 | 965571 | DNA |
| | | | | | integration/recombination/invertion |
| | | | | | protein |
| 965306-1019450 | 54144 | EEU32842.1 | 967072 | 966854 | predicted protein |
| 965306-1019450 | 54144 | EEU32843.1 | 967701 | 967090 | predicted protein |
| 965306-1019450 | 54144 | EEU32844.1 | 968005 | 967811 | predicted protein |
| 965306-1019450 | 54144 | EEU32845.1 | 968658 | 968203 | predicted protein |
| 965306-1019450 | 54144 | EEU32846.1 | 968932 | 968690 | predicted protein |
| 965306-1019450 | 54144 | EEU32847.1 | 969592 | 969347 | conserved hypothetical protein |
| 965306-1019450 | 54144 | EEU32848.1 | 969875 | 969612 | predicted protein |
| 965306-1019450 | 54144 | EEU32849.1 | 970173 | 969913 | conserved hypothetical protein |
| 965306-1019450 | 54144 | EEU32850.1 | 970620 | 970282 | conserved hypothetical protein |
| 965306-1019450 | 54144 | EEU32851.1 | 970875 | 970666 | predicted protein |
| 965306-1019450 | 54144 | EEU32852.1 | 971132 | 970980 | predicted protein |
| 965306-1019450 | 54144 | EEU32853.1 | 971450 | 971241 | predicted protein |
| 965306-1019450 | 54144 | EEU32854.1 | 971649 | 971443 | predicted protein |
| 965306-1019450 | 54144 | EEU32855.1 | 973026 | 972361 | predicted protein |
| 965306-1019450 | 54144 | EEU32856.1 | 973736 | 973035 | ATPase |
| 965306-1019450 | 54144 | EEU32857.1 | 973922 | 974233 | predicted protein |
| 965306-1019450 | 54144 | EEU32858.1 | 974562 | 974254 | predicted protein |
| 965306-1019450 | 54144 | EEU32859.1 | 975174 | 974566 | lytic transglycosylase |
| 965306-1019450 | 54144 | EEU32860.1 | 977485 | 975188 | predicted protein |
| 965306-1019450 | 54144 | EEU32861.1 | 978407 | 978042 | predicted protein |
| 965306-1019450 | 54144 | EEU32862.1 | 978937 | 978455 | conserved hypothetical protein |
| 965306-1019450 | 54144 | EEU32863.1 | 979139 | 978984 | predicted protein |
| 965306-1019450 | 54144 | EEU32864.1 | 979795 | 979139 | resolvase/recombinase |
| 965306-1019450 | 54144 | EEU32865.1 | 980528 | 979782 | conserved hypothetical protein |
| 965306-1019450 | 54144 | EEU32866.1 | 980698 | 980540 | predicted protein |
| 965306-1019450 | 54144 | EEU32867.1 | 981075 | 980707 | resolvase/recombinase |
| 965306-1019450 | 54144 | EEU32868.1 | 981730 | 981062 | conserved hypothetical protein |
| 965306-1019450 | 54144 | EEU32869.1 | 981903 | 981730 | predicted protein |
| 965306-1019450 | 54144 | EEU32870.1 | 982381 | 982019 | predicted protein |
| 965306-1019450 | 54144 | EEU32871.1 | 982653 | 982450 | predicted protein |
| 965306-1019450 | 54144 | EEU32872.1 | 983480 | 982896 | predicted protein |
| 965306-1019450 | 54144 | EEU32873.1 | 983895 | 983494 | predicted protein |
| 965306-1019450 | 54144 | EEU32874.1 | 984215 | 983892 | predicted protein |
| 965306-1019450 | 54144 | EEU32875.1 | 990789 | 984421 | helicase |
| 965306-1019450 | 54144 | EEU32876.1 | 992550 | 990865 | conserved hypothetical protein |
| 965306-1019450 | 54144 | EEU32877.1 | 993259 | 992810 | predicted protein |
| 965306-1019450 | 54144 | EEU32878.1 | 993786 | 993256 | predicted protein |
| 965306-1019450 | 54144 | EEU32879.1 | 996192 | 993802 | type I topoisomease |
| 965306-1019450 | 54144 | EEU32880.1 | 996886 | 996269 | predicted protein |
| 965306-1019450 | 54144 | EEU32881.1 | 997613 | 997140 | predicted protein |
| 965306-1019450 | 54144 | EEU32882.1 | 998631 | 997690 | predicted protein |
| 965306-1019450 | 54144 | EEU32883.1 | 999162 | 998644 | conjugative transfer signal peptidase |
| | | | | | TraF |
| 965306-1019450 | 54144 | EEU32884.1 | 999454 | 999164 | predicted protein |
| 965306-1019450 | 54144 | EEU32885.1 | 999851 | 999642 | predicted protein |
| 965306-1019450 | 54144 | EEU32886.1 | 1000454 | 1000095 | predicted protein |
| 965306-1019450 | 54144 | EEU32887.1 | 1000741 | 1000538 | predicted protein |
| 965306-1019450 | 54144 | EEU32888.1 | 1001035 | 1000859 | predicted protein |
| 965306-1019450 | 54144 | EEU32889.1 | 1001463 | 1001170 | predicted protein |
| 965306-1019450 | 54144 | EEU32890.1 | 1002564 | 1001503 | TrbL/VirB6 plasmid conjugal transfer |
| | | | | | protein |
| 965306-1019450 | 54144 | EEU32891.1 | 1003026 | 1002568 | predicted protein |
| 965306-1019450 | 54144 | EEU32892.1 | 1003896 | 1003048 | predicted protein |
| 965306-1019450 | 54144 | EEU32893.1 | 1006532 | 1004058 | conjugal transfer protein TrbE |
| 965306-1019450 | 54144 | EEU32894.1 | 1006825 | 1006538 | predicted protein |
| 965306-1019450 | 54144 | EEU32895.1 | 1007801 | 1006845 | P-type conjugative transfer ATPase |
| | | | | | TrbB |
| 965306-1019450 | 54144 | EEU32896.1 | 1008154 | 1007801 | predicted protein |
| 965306-1019450 | 54144 | EEU32897.1 | 1010223 | 1008154 | TRAG protein |
| 965306-1019450 | 54144 | EEU32898.1 | 1010784 | 1010308 | predicted protein |
| 965306-1019450 | 54144 | EEU32899.1 | 1012013 | 1010799 | conjugation TrbI family protein |
| 965306-1019450 | 54144 | EEU32900.1 | 1012811 | 1012023 | P-type conjugative transfer protein |
| | | | | | VirB9 |
| 965306-1019450 | 54144 | EEU32901.1 | 1013518 | 1012823 | conserved hypothetical protein |
| 965306-1019450 | 54144 | EEU32902.1 | 1013896 | 1013579 | conjugal transfer protein TrbC |
| 965306-1019450 | 54144 | EEU32903.1 | 1014228 | 1013893 | predicted protein |
| 965306-1019450 | 54144 | EEU32904.1 | 1015212 | 1014400 | predicted protein |
| 965306-1019450 | 54144 | EEU32905.1 | 1015636 | 1015244 | conserved hypothetical protein |
| 965306-1019450 | 54144 | EEU32906.1 | 1016055 | 1015723 | conserved hypothetical protein |
| 965306-1019450 | 54144 | EEU32907.1 | 1016469 | 1016074 | predicted protein |
| 965306-1019450 | 54144 | EEU32908.1 | 1016769 | 1016473 | conserved hypothetical protein |
| 965306-1019450 | 54144 | EEU32909.1 | 1016991 | 1016851 | conserved hypothetical protein |
| 965306-1019450 | 54144 | EEU32910.1 | 1017888 | 1017229 | conserved hypothetical protein |
| 965306-1019450 | 54144 | EEU32911.1 | 1019162 | 1017885 | predicted protein |
| 1110825-1134389 | 23564 | EEU32992.1 | 1111325 | 1110942 | toxin secretion/phage lysis holin |
| 1110825-1134389 | 23564 | EEU32993.1 | 1111789 | 1111340 | conserved hypothetical protein |
| 1110825-1134389 | 23564 | EEU32994.1 | 1112159 | 1111776 | conserved hypothetical protein |
| 1110825-1134389 | 23564 | EEU32995.1 | 1112371 | 1112174 | predicted protein |
| 1110825-1134389 | 23564 | EEU32996.1 | 1112940 | 1112431 | conserved hypothetical protein |
| 1110825-1134389 | 23564 | EEU32997.1 | 1113824 | 1113054 | conserved hypothetical protein |
| 1110825-1134389 | 23564 | EEU32998.1 | 1114533 | 1113811 | conserved hypothetical protein |
| 1110825-1134389 | 23564 | EEU32999.1 | 1115187 | 1114537 | conserved hypothetical protein |
| 1110825-1134389 | 23564 | EEU33000.1 | 1116244 | 1115180 | baseplate J-like protein |
| 1110825-1134389 | 23564 | EEU33001.1 | 1116673 | 1116245 | phage protein |
| 1110825-1134389 | 23564 | EEU33002.1 | 1117127 | 1116675 | conserved hypothetical protein |
| 1110825-1134389 | 23564 | EEU33003.1 | 1118167 | 1117124 | predicted protein |
| 1110825-1134389 | 23564 | EEU33004.1 | 1118618 | 1118172 | conserved hypothetical protein |
| 1110825-1134389 | 23564 | EEU33005.1 | 1120539 | 1118632 | phage protein |
| 1110825-1134389 | 23564 | EEU33006.1 | 1121062 | 1120601 | predicted protein |
| 1110825-1134389 | 23564 | EEU33007.1 | 1121594 | 1121226 | conserved hypothetical protein |
| 1110825-1134389 | 23564 | EEU33008.1 | 1122044 | 1121604 | conserved hypothetical protein |
| 1110825-1134389 | 23564 | EEU33009.1 | 1123136 | 1122057 | conserved hypothetical protein |
| 1110825-1134389 | 23564 | EEU33010.1 | 1123585 | 1123136 | conserved hypothetical protein |
| 1110825-1134389 | 23564 | EEU33011.1 | 1123962 | 1123582 | phage protein |
| 1110825-1134389 | 23564 | EEU33012.1 | 1124317 | 1123952 | conserved hypothetical protein |
| 1110825-1134389 | 23564 | EEU33013.1 | 1124643 | 1124314 | conserved hypothetical protein |
| 1110825-1134389 | 23564 | EEU33014.1 | 1124884 | 1124678 | conserved hypothetical protein |
| 1110825-1134389 | 23564 | EEU33015.1 | 1126087 | 1124894 | phage protein |
| 1110825-1134389 | 23564 | EEU33016.1 | 1126710 | 1126087 | predicted protein |
| 1110825-1134389 | 23564 | EEU33017.1 | 1127074 | 1126856 | conserved hypothetical protein |
| 1110825-1134389 | 23564 | EEU33018.1 | 1128740 | 1127058 | phage protein |
| 1110825-1134389 | 23564 | EEU33020.1 | 1130040 | 1128724 | terminase |
| 1110825-1134389 | 23564 | EEU33019.1 | 1130040 | 1128724 | phage portal protein |
| 1110825-1134389 | 23564 | EEU33021.1 | 1131871 | 1131479 | phage Terminase Small Subunit |
| 1110825-1134389 | 23564 | EEU33022.1 | 1132196 | 1131936 | predicted protein |
| 1110825-1134389 | 23564 | EEU33023.1 | 1133038 | 1132538 | predicted protein |
| 1110825-1134389 | 23564 | EEU33024.1 | 1133406 | 1133050 | predicted protein |
| 1110825-1134389 | 23564 | EEU33025.1 | 1134145 | 1133408 | phage antirepressor protein |
| 1110825-1134389 | 23564 | EEU33026.1 | 1134359 | 1134150 | predicted protein |
| 1134686-1141495 | 6809 | EEU33027.1 | 1136021 | 1134807 | replicative DNA helicase |
| 1134686-1141495 | 6809 | EEU33028.1 | 1136792 | 1136022 | conserved hypothetical protein |
| 1134686-1141495 | 6809 | EEU33029.1 | 1137732 | 1137460 | predicted protein |
| 1134686-1141495 | 6809 | EEU33030.1 | 1138145 | 1137930 | predicted protein |
| 1134686-1141495 | 6809 | EEU33031.1 | 1138750 | 1138265 | predicted protein |
| 1134686-1141495 | 6809 | EEU33032.1 | 1139046 | 1138882 | predicted protein |
| 1134686-1141495 | 6809 | EEU33033.1 | 1139965 | 1139114 | predicted protein |
| 1134686-1141495 | 6809 | EEU33034.1 | 1140391 | 1139975 | conserved hypothetical protein |
| 1134686-1141495 | 6809 | EEU33035.1 | 1140823 | 1140404 | predicted protein |
| 1134686-1141495 | 6809 | EEU33036.1 | 1141005 | 1141391 | predicted protein |
| 1142122-1145342 | 3220 | EEU33037.1 | 1142129 | 1142821 | conserved hypothetical protein |
| 1142122-1145342 | 3220 | EEU33038.1 | 1142831 | 1143343 | predicted protein |
| 1142122-1145342 | 3220 | EEU33039.1 | 1143353 | 1143595 | predicted protein |
| 1142122-1145342 | 3220 | EEU33040.1 | 1143607 | 1144329 | predicted protein |
| 1142122-1145342 | 3220 | EEU33041.1 | 1144326 | 1144604 | predicted protein |
| 1142122-1145342 | 3220 | EEU33042.1 | 1144729 | 1145166 | conserved hypothetical protein |
| 1142122-1145342 | 3220 | EEU33043.1 | 1145169 | 1145321 | predicted protein |
| 1145607-1149818 | 4211 | EEU33044.1 | 1145689 | 1145847 | predicted protein |
| 1145607-1149818 | 4211 | EEU33045.1 | 1145834 | 1146547 | conserved hypothetical protein |
| 1145607-1149818 | 4211 | EEU33046.1 | 1146612 | 1147022 | predicted protein |
| 1145607-1149818 | 4211 | EEU33047.1 | 1147025 | 1147480 | conserved hypothetical protein |
| 1145607-1149818 | 4211 | EEU33048.1 | 1147487 | 1147777 | predicted protein |
| 1145607-1149818 | 4211 | EEU33049.1 | 1147777 | 1148307 | conserved hypothetical protein |
| 1145607-1149818 | 4211 | EEU33050.1 | 1148298 | 1148429 | predicted protein |
| 1145607-1149818 | 4211 | EEU33051.1 | 1148500 | 1148697 | predicted protein |
| 1145607-1149818 | 4211 | EEU33052.1 | 1148672 | 1149727 | phage integrase |

De novo assembly of unmapped CC53 reads yielded 82 kbp of sequence in 67 contigs ≧500 nt. These contigs aligned with variable sequence identity to one of the sixteen Fusobacterium genome assemblies or the ATCC type strain. BLASTX (Altschul et al. 1997) searches of GenBank-nr identified 99 coding sequences (Table 4), the most recurrent of which was hemolysin, a bacterial endotoxin.

TABLE 4

| | Sequence | | | | |
|---|---|---|---|---|---|
| | identity | Alignment | Alignment | Predicted gene | |
| Accession | (%) | start | end | product | Species |
| ZP_06751356.1 | 89.8 | 2370 | 1 | Outer membrane protein | Fusobacterium |
| | | | | | sp. 3_1_27 |
| ZP_00144646.1 | 99.6 | 39 | 1658 | Oligopeptide-binding | Fusobacterium |
| | | | | protein oppA | nucleatum |
| | | | | | subsp. |
| | | | | | vincentii |
| | | | | | ATCC 49256 |
| ZP_00144647.1 | 99.4 | 1695 | 2702 | Dipeptide transport | Fusobacterium |
| | | | | system permease protein | nucleatum |
| | | | | dppB | subsp. |
| | | | | | vincentii |
| | | | | | ATCC 49256 |
| ZP_00144648.1 | 100.0 | 2737 | 3531 | Oligopeptide transport | Fusobacterium |
| | | | | system permease protein | nucleatum |
| | | | | oppC | subsp. |
| | | | | | vincentii |
| | | | | | ATCC 49256 |
| ZP_00144649.1 | 99.4 | 3543 | 4079 | Oligopeptide transport | Fusobacterium |
| | | | | ATP-binding protein | nucleatum |
| | | | | oppD | subsp. |
| | | | | | vincentii |
| | | | | | ATCC 49256 |
| ZP_00144649.1 | 49.1 | 4322 | 4780 | Oligopeptide transport | Fusobacterium |
| | | | | ATP-binding protein | nucleatum |
| | | | | oppD | subsp. |
| | | | | | vincentii |
| | | | | | ATCC 49256 |
| ZP_00144515.1 | 100.0 | 1 | 255 | Hypothetical Membrane | Fusobacterium |
| | | | | Spanning Protein | nucleatum |
| | | | | | subsp. |
| | | | | | vincentii |
| | | | | | ATCC 49256 |
| ZP_05552202.1 | 100.0 | 276 | 518 | Conserved hypothetical | Fusobacterium |
| | | | | protein | sp. 3_1_36A2 |
| ZP_04574788.1 | 100.0 | 48 | 1598 | Propionate CoA- | Fusobacterium |
| | | | | transferase | sp. 7_1 |
| ZP_04574789.1 | 100.0 | 1677 | 3014 | Propionate permease | Fusobacterium |
| | | | | | sp. 7_1 |
| ZP_04571805.1 | 95.8 | 2 | 427 | Hemolysin | Fusobacterium |
| | | | | | sp. 4_1_13 |
| YP_003945856.1 | 82.6 | 458 | 733 | Hemagglutination | Paenibacillus |
| | | | | activity domain protein | polymyxa SC2 |
| ZP_04571802.1 | 90.9 | 1075 | 1305 | Conserved hypothetical | Fusobacterium |
| | | | | protein | sp. 4_1_13 |
| ZP_00144656.1 | 100.0 | 1337 | 1519 | Hypothetical protein | Fusobacterium |
| | | | | | nucleatum |
| | | | | | subsp. |
| | | | | | vincentii |
| | | | | | ATCC 49256 |
| ZP_00144284.1 | 99.6 | 1343 | 3 | Hemolysin | Fusobacterium |
| | | | | | nucleatum |
| | | | | | subsp. |
| | | | | | vincentii |
| | | | | | ATCC 49256 |
| ZP_05551287.1 | 100.0 | 485 | 3 | Histidyl-tRNA | Fusobacterium |
| | | | | synthetase | sp. 3_1_36A2 |
| ZP_05442395.1 | 100.0 | 907 | 500 | Recombination factor | Fusobacterium |
| | | | | protein RarA | sp. D11 |
| ZP_00143333.1 | 99.4 | 1797 | 238 | Hypothetical protein | Fusobacterium |
| | | | | | nucleatum |
| | | | | | subsp. |
| | | | | | vincentii |
| | | | | | ATCC 49256 |
| ZP_05550632.1 | 97.9 | 1933 | 1793 | Conserved hypothetical | Fusobacterium |
| | | | | protein | sp. 3_1_36A2 |
| ZP_06750290.1 | 99.7 | 1685 | 693 | ABC transporter iron | Fusobacterium |
| | | | | chelate uptake | sp. 3_1_27 |
| | | | | transporter (FeCT) | |
| | | | | family | |
| ZP_06869931.1 | 99.4 | 2753 | 1704 | Iron(III) dicitrate- | Fusobacterium |
| | | | | binding protein | nucleatum |
| | | | | | subsp. |
| | | | | | nucleatum |
| | | | | | ATCC 23726 |
| ZP_05816109.1 | 98.9 | 4431 | 2779 | Conserved hypothetical | Fusobacterium |
| | | | | protein | sp. 3_1_33 |
| ZP_06750286.1 | 99.6 | 5210 | 4473 | Nitrogenase iron protein | Fusobacterium |
| | | | | | sp. 3_1_27 |
| NP_603209.1 | 99.2 | 6017 | 5295 | Iron(III) dicitrate | Fusobacterium |
| | | | | transport ATP-binding | nucleatum |
| | | | | protein fecE | subsp. |
| | | | | | nucleatum |
| | | | | | ATCC 25586 |
| ZP_04571805.1 | 99.4 | 1515 | 1 | Hemolysin | Fusobacterium |
| | | | | | sp. 4_1_13 |
| ZP_06749749.1 | 97.2 | 1072 | 2 | Hemolysin | Fusobacterium |
| | | | | | sp. 3_1_27 |
| ZP_04572815.1 | 99.8 | 1997 | 465 | Dipeptide-binding | Fusobacterium |
| | | | | protein | sp. 4_1_13 |
| ZP_05816112.1 | 100.0 | 1435 | 2007 | Iron(III) dicitrate | Fusobacterium |
| | | | | transport system | sp. 3_1_33 |
| | | | | permease fecD | |
| ZP_05816113.1 | 99.1 | 372 | 1340 | Iron(III) dicitrate- | Fusobacterium |
| | | | | binding protein | sp. 3_1_33 |
| ZP_04572103.1 | 99.6 | 1 | 1491 | Conserved hypothetical | Fusobacterium |
| | | | | protein | sp. 4_1_13 |
| ZP_00144407.1 | 100.0 | 1746 | 2363 | Threonyl-tRNA | Fusobacterium |
| | | | | synthetase | nucleatum |
| | | | | | subsp. |
| | | | | | vincentii |
| | | | | | ATCC 49256 |
| ZP_00144406.1 | 100.0 | 517 | 20 | Hypothetical protein | Fusobacterium |
| | | | | | nucleatum |
| | | | | | subsp. |
| | | | | | vincentii |
| | | | | | ATCC 49256 |
| ZP_00144407.1 | 100.0 | 1072 | 551 | Threonyl-tRNA | Fusobacterium |
| | | | | synthetase | nucleatum |
| | | | | | subsp. |
| | | | | | vincentii |
| | | | | | ATCC 49256 |
| ZP_06751328.1 | 100.0 | 475 | 248 | Conserved hypothetical | Fusobacterium |
| | | | | protein | sp. 3_1_27 |
| ZP_04571297.1 | 100.0 | 79 | 216 | Conserved hypothetical | Fusobacterium |
| | | | | protein | sp. 4_1_13 |
| ZP_04971343.1 | 96.6 | 446 | 3 | Short-chain alcohol | Fusobacterium |
| | | | | dehydrogenase | nucleatum |
| | | | | | subsp. |
| | | | | | polymorphum |
| | | | | | ATCC 10953 |
| ZP_06750238.1 | 100.0 | 1030 | 614 | Tetratricopeptide repeat | Fusobacterium |
| | | | | family protein | sp. 3_1_27 |
| ZP_00143341.1 | 100.0 | 1 | 819 | Hypothetical protein; | Fusobacterium |
| | | | | Spore photoproduct | nucleatum |
| | | | | lyase | subsp. |
| | | | | | vincentii |
| | | | | | ATCC 49256 |
| ZP_04571419.1 | 100.0 | 550 | 353 | Outer membrane protein | Fusobacterium |
| | | | | | sp. 4_1_13 |
| ZP_02212678.1 | 85.2 | 244 | 2 | Hypothetical protein | Clostridium |
| | | | | CLOBAR_02295 | bartlettii DSM |
| | | | | | 16795 |
| ZP_04971346.1 | 96.3 | 406 | 567 | Hypothetical protein | Fusobacterium |
| | | | | FNP_1655 | nucleatum |
| | | | | | subsp. |
| | | | | | polymorphum |
| | | | | | ATCC 10953 |
| ZP_04572112.1 | 100.0 | 1000 | 287 | 2-nitropropane | Fusobacterium |
| | | | | dioxygenase | sp. 4_1_13 |
| ZP_00143874.1 | 93.9 | 736 | 638 | Hypothetical protein | Fusobacterium |
| | | | | | nucleatum |
| | | | | | subsp. |
| | | | | | vincentii |
| | | | | | ATCC 49256 |
| ZP_06750274.1 | 99.0 | 3 | 599 | Conserved hypothetical | Fusobacterium |
| | | | | protein | sp. 3_1_27 |
| ZP_05442397.1 | 100.0 | 620 | 922 | Hypothetical protein | Fusobacterium |
| | | | | PrD11_11325 | sp. D11 |
| ZP_06751048.1 | 100.0 | 935 | 3 | Formimidoylglutamase | Fusobacterium |
| | | | | | sp. 3_1_27 |
| ZP_04572326.1 | 99.6 | 1730 | 2545 | Polysialic acid capsule | Fusobacterium |
| | | | | expression protein kpsF | sp. 4_1_13 |
| ZP_06749764.1 | 95.0 | 545 | 72 | Conserved hypothetical | Fusobacterium |
| | | | | protein | sp. 3_1_27 |
| ZP_05631047.1 | 80.4 | 1017 | 559 | Hypothetical protein | Fusobacterium |
| | | | | FgonA2_04800 | gonidiaformans |
| | | | | | ATCC |
| | | | | | 25563 |
| ZP_06749761.1 | 100.0 | 1399 | 1106 | Hemolysin | Fusobacterium |
| | | | | | sp. 3_1_27 |
| ZP_05440307.1 | 100.0 | 1 | 282 | Uroporphyrinogen-III | Fusobacterium |
| | | | | synthase | sp. D11 |
| ZP_00143146.1 | 98.9 | 528 | 1 | Serine protease | Fusobacterium |
| | | | | | nucleatum |
| | | | | | subsp. |
| | | | | | vincentii |
| | | | | | ATCC 49256 |
| ZP_04572999.1 | 100.0 | 1 | 555 | ATP-NAD kinase | Fusobacterium |
| | | | | | sp. 4_1_13 |
| ZP_06750251.1 | 100.0 | 537 | 905 | DNA repair protein | Fusobacterium |
| | | | | RecN | sp. 3_1_27 |
| ZP_00144496.1 | 98.3 | 1038 | 1 | Outer membrane protein | Fusobacterium |
| | | | | family | nucleatum |
| | | | | | subsp. |
| | | | | | vincentii |
| | | | | | ATCC 49256 |
| ZP_00144049.1 | 99.6 | 2 | 679 | 4- | Fusobacterium |
| | | | | hydroxybutyrate:acetyl- | nucleatum |
| | | | | CoA CoA transferase | subsp. |
| | | | | | vincentii |
| | | | | | ATCC 49256 |
| ZP_06750263.1 | 100.0 | 636 | 1 | tRNA (guanine-N1)- | Fusobacterium |
| | | | | methyltransferase | sp. 3_1_27 |
| ZP_04572837.1 | 100.0 | 180 | 515 | Ribosomal-protein- | Fusobacterium |
| | | | | alanine acetyltransferase | sp. 4_1_13 |
| ZP_04574766.1 | 100.0 | 273 | 881 | Methyltransferase | Fusobacterium |
| | | | | | sp. 7_1 |
| ZP_05551301.1 | 100.0 | 123 | 680 | Conserved hypothetical | Fusobacterium |
| | | | | protein | sp. 3_1_36A2 |
| ZP_00144645.1 | 100.0 | 434 | 670 | Hypothetical protein | Fusobacterium |
| | | | | | nucleatum |
| | | | | | subsp. |
| | | | | | vincentii |
| | | | | | ATCC 49256 |
| ZP_04571805.1 | 97.3 | 2 | 2755 | Hemolysin | Fusobacterium |
| | | | | | sp. 4_1_13 |
| ZP_06751292.1 | 99.5 | 3 | 569 | Dipeptide-binding | Fusobacterium |
| | | | | protein | sp. 3_1_27 |
| ZP_04574649.1 | 100.0 | 631 | 29 | Membrane protein | Fusobacterium |
| | | | | | sp. 7_1 |
| ZP_00143639.1 | 99.7 | 2 | 967 | Aspartate | Fusobacterium |
| | | | | aminotransferase | nucleatum |
| | | | | | subsp. |
| | | | | | vincentii |
| | | | | | ATCC 49256 |
| ZP_04574784.1 | 99.6 | 463 | 1143 | Conserved hypothetical | Fusobacterium |
| | | | | protein | sp. 7_1 |
| ZP_05550411.1 | 99.5 | 598 | 2 | D-methionine ABC | Fusobacterium |
| | | | | transporter, ATP- | sp. 3_1_36A2 |
| | | | | binding protein | |
| ZP_06524412.1 | 99.3 | 1001 | 180 | Export ABC transporter | Fusobacterium |
| | | | | | sp. D11 |
| ZP_04574771.1 | 100.0 | 2 | 652 | Riboflavin kinase | Fusobacterium |
| | | | | | sp. 7_1 |
| ZP_05814256.1 | 100.0 | 627 | 1301 | Conserved hypothetical | Fusobacterium |
| | | | | protein | sp. 3_1_33 |
| ZP_04572332.1 | 100.0 | 1044 | 151 | Conserved hypothetical | Fusobacterium |
| | | | | protein | sp. 4_1_13 |
| ZP_06750860.1 | 99.4 | 3 | 941 | Conserved hypothetical | Fusobacterium |
| | | | | protein | sp. 3_1_27 |
| ZP_04572817.1 | 98.9 | 3 | 782 | Polysaccharide | Fusobacterium |
| | | | | deacetylase | sp. 4_1_13 |
| ZP_04970650.1 | 98.3 | 178 | 2 | Possible DNA repair | Fusobacterium |
| | | | | photolyase | nucleatum |
| | | | | | subsp. |
| | | | | | polymorphum |
| | | | | | ATCC 10953 |
| ZP_00143340.1 | 100.0 | 475 | 206 | Hypothetical protein | Fusobacterium |
| | | | | | nucleatum |
| | | | | | subsp. |
| | | | | | vincentii |
| | | | | | ATCC 49256 |
| ZP_00143339.1 | 100.0 | 749 | 471 | Hypothetical protein | Fusobacterium |
| | | | | | nucleatum |
| | | | | | subsp. |
| | | | | | vincentii |
| | | | | | ATCC 49256 |
| ZP_05816107.1 | 100.0 | 2 | 400 | Iron(III) dicitrate | Fusobacterium |
| | | | | transport system | sp. 3_1_33 |
| | | | | permease fecD | |
| ZP_04572103.1 | 100.0 | 1 | 666 | Conserved hypothetical | Fusobacterium |
| | | | | protein | sp. 4_1_13 |
| ZP_04572180.1 | 99.1 | 978 | 304 | Filamentation induced | Fusobacterium |
| | | | | by cAMP protein Fic | sp. 4_1_13 |
| ZP_06871387.1 | 100.0 | 3 | 395 | Oxidoreductase | Fusobacterium |
| | | | | | nucleatum |
| | | | | | subsp. |
| | | | | | nucleatum |
| | | | | | ATCC 23726 |
| ZP_04572329.1 | 100.0 | 768 | 466 | Conserved hypothetical | Fusobacterium |
| | | | | protein | sp. 4_1_13 |
| ZP_05550650.1 | 100.0 | 1220 | 810 | Glycerol-3-phosphate | Fusobacterium |
| | | | | dehydrogenase | sp. 3_1_36A2 |
| | | | | (NAD(+)) | |
| ZP_04572735.1 | 100.0 | 2 | 526 | Uracil-DNA glycosylase | Fusobacterium |
| | | | | | sp. 4_1_13 |
| ZP_06750465.1 | 100.0 | 1482 | 667 | Sensory Transduction | Fusobacterium |
| | | | | Protein Kinase | sp. 3_1_27 |
| ZP_06749809.1 | 99.6 | 707 | 6 | Methylaspartate mutase | Fusobacterium |
| | | | | E subunit | sp. 3_1_27 |
| ZP_06748447.1 | 100.0 | 1 | 243 | ATP synthase F1 beta | Fusobacterium |
| | | | | subunit | sp. |
| | | | | | 1_1_41FAA |
| ZP_00144388.1 | 100.0 | 256 | 552 | ATP synthase epsilon | Fusobacterium |
| | | | | chain sodium ion | nucleatum |
| | | | | specific | subsp. |
| | | | | | vincentii |
| | | | | | ATCC 49256 |
| ZP_04572312.1 | 100.0 | 610 | 293 | Dihydrolipoamide | Fusobacterium |
| | | | | acyltransferase | sp. 4_1_13 |
| ZP_06750801.1 | 98.6 | 3 | 626 | NAD(FAD)-utilizing | Fusobacterium |
| | | | | dehydrogenase | sp. 3_1_27 |
| ZP_05814379.1 | 100.0 | 580 | 2 | Membrane protein | Fusobacterium |
| | | | | | sp. 3_1_33 |
| ZP_04572122.1 | 98.6 | 3 | 434 | RNA polymerase sigma- | Fusobacterium |
| | | | | 54 factor rpoN | sp. 4_1_13 |
| ZP_00144056.1 | 100.0 | 842 | 498 | Bacterial/Archaeal | Fusobacterium |
| | | | | Transporter family | nucleatum |
| | | | | protein | subsp. |
| | | | | | vincentii |
| | | | | | ATCC 49256 |
| ZP_06750657.1 | 100.0 | 3 | 368 | branched-chain amino | Fusobacterium |
| | | | | acid transport system II | sp. 3_1_27 |
| | | | | carrier protein | |
| ZP_04572207.1 | 99.0 | 599 | 3 | Ribosomal large subunit | Fusobacterium |
| | | | | pseudouridine synthase B | sp. 4_1_13 |
| ZP_00144400.1 | 98.0 | 2 | 295 | Hypothetical protein | Fusobacterium |
| | | | | | nucleatum |
| | | | | | subsp. |
| | | | | | vincentii |
| | | | | | ATCC 49256 |
| ZP_04572112.1 | 100.0 | 376 | 708 | 2-nitropropane | Fusobacterium |
| | | | | dioxygenase | sp. 4_1_13 |
| ZP_04574788.1 | 100.0 | 2 | 628 | Propionate CoA- | Fusobacterium |
| | | | | transferase | sp. 7_1 |
| ZP_04572174.1 | 99.1 | 7 | 660 | WD-repeat family | Fusobacterium |
| | | | | protein | sp. 4_1_13 |

Although we were able to culture Fusobacterium from only a single tumour section, we used primer walking to interrogate an additional four samples where qPCR-predicted levels of Fusobacterium were high.

More specifically, PCR primers were designed using primer 3.0 and the F. nucleatum types strain (ATCC 25586) genome as reference. For PCR, 1 ng of extracted gDNA was used as template, Phusion polymerase (NEB) and buffers were used for the PCR. Cycling conditions were as follows: 94° C. for 2 minutes, then 94° C. 30 seconds, 67° C. 30 seconds, 72° C. 30 seconds for 30 cycles. PCR products were purified using Ampure magnetic beads. Sequencing reactions were done using BigDye 3.1 and reaction products were run on AB 3730xl. Phred quality 30 trimmed sequences were used in a BLASTN alignment against the HMP reference genome data, keeping the hit with the highest sequence identity.

Sanger sequences from these amplicons comprised 68,694 total base pairs and each aligned with highest sequence similarity (93-100%) to one of the various Fusobacterium draft genomes, although we could not assign unambiguously a specific best matching strain to any of these samples, due perhaps to within-sample strain heterogeneity.

### Example 4

### CC53 Demonstrates Invasiveness in Human Colonic Epithelial Cells

We were interested to determine if CC53 would demonstrate invasiveness in human colonic epithelial cells. We used immunofluorescence and an antibody-based differential staining method, described previously (Strauss et al. 2011), to measure invasion of cultured colonic adenocarcinoma-2 (Caco-2) cells by the Fusobacterium tumour isolate. Caco-2 cells were grown on glass coverslips, infected with CC53 culture (at a multiplicity of infection of 100:1), and then differentially stained with anti-Fusobacterium antibodies conjugated to different fluorophores before and after Caco-2 cell permeabilization.

More specifically, Caco-2 cell invasion assays with CC53 were carried out in triplicate using a differential staining immunofluorescence procedure. Briefly, bacterial cultures were grown to late log phase according to pre-determined growth-curve data, and normalized for cell number using McFarland standards. Caco-2 cells were grown to 80% confluence on glass coverslips in 24-well plates and infected at a multiplicity of infection of 100:1 (bacterial cells:intestinal cells). Infected cells were maintained at 37° C., 5% CO2 for 4 hours following infection, after which time cells were washed with PBS to remove non-adherent bacteria, and then fixed with 2.5% paraformaldehyde, and blocked in 10% (v/v) normal goat serum. Prepared polyclonal antibodies were diluted to 1/500, applied to coverslips, and incubated for 1 hr at 37° C. Coverslips were then incubated with donkey anti-rabbit (EAV_AS1) or anti-rat (EAV_AS2) Alexa 350 (1/100) (Molecular Probes), permeabilized by the addition of 0.1% TritonX100, and then reincubated with prepared polyclonal antibodies, as above. Following this, cells were labeled with donkey anti rat or anti-rabbit Cy3 (1/500) for 30 mins at 37° C., as well as Alexa 488 Phalloidin (Molecular Probes) (1/200). Coverslips were mounted onto glass slides and examined at 40× magnification using a Leica DMIREB2 microscope and an ORCA-ER digital camera. Images were captured using Volocity (Improvision) software.

The differential staining method allows for delineation between bacteria that have penetrated the host cells (labeled for actin) to reside within them, and bacteria present on the outside of the cell. Using this protocol, bacteria external to the host cell were labeled with both Cy3 and Alexa 350, whereas bacteria inside the cells were labeled with Cy3 only (appearing only orange when channels were merged). Each invasion assay was carried out on 3 separate occasions using freshly prepared Caco-2 cells and bacterial inocula. CC53 shows a very long, fine, thread-like cell morphology and, in our study, the long, thread-like cells appear to penetrate host cells pole-first and demonstrate a very long, flexible cell morphology. This assay demonstrated that CC53 was invasive.

### Example 5

### Clinical Correlates of Fusobacterium Overabundance

We explored clinical correlates of Fusobacterium overabundance and, in this study, did not observe any association with tumour stage, tumour site, history of treatment, patient age or survival. To explore histopathological correlates, an H&E stained section from a representative cross section clinical block from each tumour was scored for lymphocytic infiltrates, myeloid/neutrophil infiltrates, circumferential involvement, and luminal or geographic necrosis, and these scores were compared to Fusobacterium relative abundance (tumour versus control). Fusobacterium showed higher relative abundance in tumours with >50% circumferential involvement (unpaired, two-tailed t-test, p=0.0023). In addition, we found that subjects with high relative abundance Fusobacterium in tumour relative to matched control tissue were significantly more likely to have regional lymph node metastases, as determined by their TNM scores (one-tailed Fisher's exact test, p=0.0035) (FIG. 5). Specifically, lymph node metastases were present in 29/39 patients in the high abundance Fusobacterium group versus 26/58 in the low abundance group.

### REFERENCES

Altschul, S. and Miller, W. 1997. Gapped BLAST and PSI-BLAST: a new generation of protein database search programs. Nucleic Acids Res. 25: 3389-3402.
Ben-Dov, E. and Kushmaro, A. 2006. Advantage of using inosine at the 3′ termini of 16S rRNA gene universal primers for the study of microbial diversity. Appl. Environ. Microbiol. 72: 6902-6906.
Bolstad, A., Jensen, H., and Bakken, V. 1996. Taxonomy, biology, and periodontal aspects of Fusobacterium nucleatum. Clin. Microbiol. Rev. 9: 55-71.
Ewing, B. and Green, P. 1998. Base-calling of automated sequencer traces using phred. I. Accuracy assessment. Genome Res. 8: 175-185.
Flicek P, Amode M R, Barrell D, Beal K, Brent S, Chen Y, Clapham P, Coates G, Fairley S, Fitzgerald S et al. 2011. Ensembl 2011. Nucleic Acids Res 39: D800-D806.
Han, Y. W., Shi, W., Huang, G. T., Kinder Haake, S., Park, N. H., Kuramitsu, H. and Genco, R. J. 2000. Interactions between periodontal bacteria and human oral epithelial cells: Fusobacterium nucleatum adheres to and invades epithelial cells. Infect. Immun. 68:3140-3146.
Han, X., Weinberg, J., Prabhu, S., Hassenbusch, S., Fuller, G., and Tarrand, J. 2003. Fusobacterial brain abscess: a review of five cases and an analysis of possible pathogenesis. J. Neurosurg. 99: 693-700.
Herrera L A et al. Role of infectious diseases in human carcinogenesis. Environmental & Molecular Mutagenesis. 2005; 45 (284-303)).
Kai, A., Cooke, F., Antoun, N., Siddharthan, C., and Sule, O. 2008. A rare presentation of ventriculitis and brain abscess caused by Fusobacterium nucleatum. J. Med. Microbiol. 57: 668-671.
Kim M-K, Kim H-K, Kim B-O, Yoo S Y, Seong J-H, Kim D-K, Lee S E, Choe S-J, Park J-C, Min B-M et al. 2004. Multiplex PCR using conserved and species-specific 16S rDNA primers for simultaneous detection of Fusobacterium nucleatum and Actinobacillus actinomycetemcomitans. J Microbiol Biotechnol 14: 110-115.
Krisanaprakornkit, S. and Dale, D. 2000. Inducible expression of human beta-defensin 2 by Fusobacterium nucleatum in oral epithelial cells: Multiple signaling pathways and role of commensal bacteria in innate immunity and the epithelial barrier. Infect. Immun. 68: 2907-2915.
Li, H. and Durbin, R. 2010. Fast and accurate long-read alignment with Burrows-Wheeler transform. Bioinformatics 26: 589-595.
Marshall, B. and Warren, J. 1984. Unidentified curved bacilli in the stomach of patients with gastritis and peptic-ulceration. Lancet 1: 1311-1315.
McLean M H, Murray G I, Stewart K N, Norrie G, Mayer C, Hold G L, Thomson J, Fyfe N, Hope M, Mowat N A et al. 2011. The Inflammatory Microenvironment in Colorectal Neoplasia. PLoS One 6: e15366.
Moore, R. A., Warren, R. L., Freeman, J. D., Gustaysen, J. A., Chénard, C., Friedman, J. M., Suttle, C. A., Zhao, Y., and Holt, R. A. 2011. The Sensitivity of Massively Parallel Sequencing for Detecting Candidate Infectious Agents Associated with Human Tissue. PLoS One 6: e19838.
Morin R D, Johnson N A, Severson T M, Mungall A J, An J, Goya R, Paul J E, Boyle M, Woolcock B W, Kuchenbauer F et al. 2010. Somatic mutations altering EZH2 (Tyr641) in follicular and diffuse large B-cell lymphomas of germinal-center origin. Nat Genet 42: 181-185.
Nelson K E, Weinstock G M, Highlander S K, Worley K C, Creasy H H, Wortman J R, Rusch D B, Mitreva M, Sodergren E et al. 2010. A Catalog of Reference Genomes from the Human Microbiome. Science 328: 994-999.
Parkin, D. 2006. The global health burden of infection-associated cancers in the year 2002. Int. J. Cancer 118: 3030-3044.
Peyret-Lacombe, A., Brunel, G., Watts, M., Charveron, M., and Duplan, H. 2009. TLR2 sensing of F. nucleatum and S. sanguinis distinctly triggered gingival innate response. Cytokine 46: 201-210.
Qin J, Li R, Raes J, Arumugam M, Burgdorf K S, Manichanh C, Nielsen T, Pons N, Levenez F, Yamada T et al. 2010. A human gut microbial gene catalogue established by metagenomic sequencing. Nature 464:59-65.
Shah S P, Morin R D, Khattra J, Prentice L, Pugh T, Burleigh A, Delaney A, Gelmon K, Guliany R, Senz J et al. 2009. Mutational evolution in a lobular breast tumour profiled at single nucleotide resolution. Nature 461: 809-813.
Signat, B. and Duffaut, D. 2011. Role of Fusobacterium nucleatum in Periodontal Health and Disease. Curr. Issues Mol. Biol. 13: 25-35.
Sokal, R. and Michener, C. 1958. A statistical method for evaluating systematic relationships. Univ. Kans. Sci. Bull. 28: 1409-1438.
Strauss, J., Kaplan, G. G., Beck, P. L., Rioux, K., Panaccione, R., DeVinney, R., Lynch, T., and Allen-Vercoe, E. 2011. Invasive potential of gut mucosa-derived Fusobacterium nucleatum positively correlates with IBD status of the host. Inflammatory Bowel Diseases. Advance online Publication.
Strauss, J., White, A., Ambrose, C., McDonald, J., and Allen-Vercoe, E. 2008. Phenotypic and genotypic analyses of clinical Fusobacterium nucleatum and Fusobacterium periodonticum isolates from the human gut. Anaerobe 14: 301-309.
Swidsinski, A. and Ismail, M. 2011. Acute appendicitis is characterised by local invasion with Fusobacterium nucleatum/necrophorum. Gut 60: 34-40.
Vogelstein, B. and Leppert, M. 1988. Genetic alterations during colorectal-tumour development. N. Engl. J. Med. 319: 525-532.
Warren, R. and Holt, R. 2007. Assembling millions of short DNA sequences using SSAKE. Bioinformatics 23: 500-501.
Watson, P. H. 2010. The BC Cancer Agency Tumour Tissue Repository. Biopreserv. Biobanking 8: 2.
Weber, G., Shendure, J., Tanenbaum, D. M., Church, G. M., and Meyerson, M. 2002. Identification of foreign gene sequences by transcript filtering against the human genome. Nat. Genet. 30: 141-142.
Weeks, D. F., Katz, D. S., Saxon, P. and Kubal, W. S. Lemierre syndrome: report of five new cases and literature review. 2010 Emerg. Radiol. 17:323-328.
Weiss, E. and Metzger, Z. 2000. Attachment of Fusobacterium nucleatum PK1594 to mammalian cells and its coaggregation with periodontopathogenic bacteria are mediated by the same galactose-binding adhesin. Oral Microbiol. Immunol. 15: 371-377.
Wilson, G., Flibotte, S., Chopra, V., Melnyk, B., Honer, W., and Holt, R. 2006. DNA copy-number analysis in bipolar disorder and schizophrenia reveals aberrations in genes involved in glutamate signaling. Hum. Mol. Genet. 15: 743-749.
Wu S, Rhee K J, Albesiano E, Rabizadeh S, Wu X, Yen H R, Huso D L, Brancati F L, Wick E, McAllister F et al. 2009. A human colonic commensal promotes colon tumourigenesis via activation of T helper type 17 T cell responses. Nat Med 15: 1016-1022.
Ximenez-Fyvie, L. and Socransky, S. 2000. Comparison of the microbiota of supra- and subgingival plaque in health and periodontitis. J. Clin. Periodontol. 27: 648-657.

All citations are hereby incorporated by reference.

The present invention has been described with regard to one or more embodiments. However, it will be apparent to persons skilled in the art that a number of variations and modifications can be made without departing from the scope of the invention as defined in the claims.

1. A method for prognosing or diagnosing a gastrointestinal cancer in a subject, the method comprising:
a) providing a sample from the subject; and
b) detecting a Fusobacterium sp. in the sample,
wherein a positive detection of the Fusobacterium sp. indicates a prognosis or diagnosis of gastrointestinal cancer.

2. The method of claim 1 wherein the detecting comprises contacting the sample with:
a) an antibody that specifically binds a Fusobacterium sp. antigen or
b) a nucleotide sequence that hybridizes to a Fusobacterium sp. nucleotide sequence,
wherein the specific binding of the antibody to the Fusobacterium sp. antigen or the hybridization of the nucleotide sequence to the Fusobacterium sp. nucleotide sequence indicates a prognosis or diagnosis of gastrointestinal cancer.

3. The method of claim 2, wherein the Fusobacterium sp. antigen is selected from the group consisting of one or more of the polypeptides set forth in Table 4.

4. The method of claim 2, wherein the Fusobacterium sp. nucleotide sequence is selected from the group consisting of one or more of the sequences set forth in Tables 2 or 4.

5. The method of claim 1, wherein the gastrointestinal cancer is a colorectal carcinoma.

6. The method claim 1, wherein the subject has or is suspected of having chronic inflammatory bowel disease.

7. The method of one of claim 1, wherein the sample is a colon sample, a rectal sample, or a stool sample.

8. The method of one of claim 1, wherein the sample is an adenomatous lesion or polyp.

9. The method of one of claim 1, wherein the Fusobacterium sp. is a F. nucleatum.

10. A method of screening for a compound for treating a gastrointestinal cancer, the method comprising:
a) providing a test compound; and
b) determining whether the test compound inhibits the growth or activity of a Fusobacterium sp.,
wherein a compound that inhibits the growth or activity of the Fusobacterium sp. is a candidate compound for treating a gastrointestinal cancer.

11. A method of treating a gastrointestinal cancer, the method comprising administering a compound or composition that induces an immunological response against a Fusobacterium sp. to a subject diagnosed with or suspected of having a gastrointestinal cancer.

12. The method of claim 1, wherein the subject is a human.
